(19) United States
(12) Patent Application Publication
(10) Pub. No.: US 2023/0416364 A1
(43) Pub. Date: Dec. 28, 2023

(54) METHODS OF REDIRECTING OF IL-2 TO TARGET CELLS OF INTEREST

(71) Applicant: BRISTOL-MYERS SQUIBB COMPANY, Princeton, NJ (US)

(72) Inventors: Flavio SCHWARZ, San Francisco, CA (US); Xiaodi DENG, San Mateo, CA (US); Pavel STROP, San Mateo, CA (US)

(21) Appl. No.: 18/041,433

(22) PCT Filed: Aug. 12, 2021
(86) PCT No.: PCT/US2021/045718

§ 371 Date: Feb. 13, 2023

Related U.S. Application Data
(60) Provisional application No. 63/065,275, filed on Aug. 13, 2020.

(51) Int. Cl. C07K 16/28; C12N 15/86; C07K 14/715; A61K 38/20; A61P 35/00

(52) U.S. Cl. CPC C07K 16/2818; C12N 15/86; C07K 14/715; A61K 38/00; A61P 35/00; C12N 2740/15043; C07K 2319/30; A61K 38/2013

(57) ABSTRACT

The present disclosure provides constructs comprising an anti-PDI antibody, or an alternative targeting moiety, fused to CD25 or an IL-2 binding fragment of CD25. Such constructs find use in treating human diseases, such as cancer.

### CROSS REFERENCE TO RELATED APPLICATIONS

This application claims priority to U.S. Provisional Application No. 63/065,275 filed 13 Aug. 2020, the disclosure of which is incorporated herein by reference.

### SEQUENCE LISTING

The Sequence Listing filed electronically herewith is also hereby incorporated by reference in its entirety (File Name: 20210809_SEQL_13390WOPCT_GB.txt; Date Created: 9 Aug. 2021; File Size: 142 KB).

### BACKGROUND OF THE INVENTION

The immune system is capable of controlling tumor development and mediating tumor regression. Immune activating molecules, such as interleukin 2 (IL-2), can enhance anti-tumor immunity but can simultaneously lead to generalized immune activation and dose-limiting side effects. Aldesleukin (PROLEUKIN®), a slightly modified human IL-2 polypeptide, was first approved in the United States in 1998 for treatment of advanced and metastatic melanoma, but its use has been limited by toxicity issues, which are illustrated by the full-page black box warning on its prescribing information. As a cytokine, it also exhibits a short half-life (less than two hours) necessitating dosing multiple cycles of intravenous administration three times a day (TID) for five days in a row.

The need exists for improved methods of enhancing anti-tumor immune response that preferentially amplify naturally existing anti-tumor immune response against the tumor, without amplifying systemic effects leading to toxic side effects.

### SUMMARY OF THE INVENTION

The present invention provides polypeptide constructs comprising a targeting moiety and a CD25 moiety. In various embodiments, the targeting moiety binds to PD-1, NKG2a, CD8a, FcRL6, CRTAM or LAG3, such as an antibody raised against one of these targets or an antigen binding fragment thereof. In one embodiment, the invention provides polypeptide constructs comprising a PD-1 binding moiety, such as an anti-PD-1 antibody or an antigen binding fragment thereof, and a CD25 moiety.

In some embodiments, the PD-1 binding moiety in the construct comprise an anti-PD-1 antibody or antigen fragment thereof, such as an anti-mouse PD-1 antibody (e.g. mAb 4H2) or an antigen fragment thereof, or an anti-human PD-1 antibody (e.g. nivolumab or pembrolizumab) or an antigen fragment thereof. In some embodiments the PD-1 moiety comprises the heavy and light chain sequences of anti-mouse mAb 4H2 (SEQ ID NOs: 5 and 6). In other embodiments the PD-1 moiety comprises the CDRs of nivolumab (SEQ ID NOs: 17-22), the heavy and light chain variable domain sequences of nivolumab (SEQ ID NOs: 23 and 24), or the heavy and light chain sequences of nivolumab (SEQ ID NOs: 25 and 27). In further embodiments the PD-1 moiety comprises the CDRs of pembrolizumab (SEQ ID NOs: 36-41), the heavy and light chain variable domain sequences of pembrolizumab (SEQ ID NOs: 42 and 43), or the heavy and light chain sequences of pembrolizumab (SEQ ID NOs: 44 and 46).

In some embodiments, the PD-1 binding moiety in the construct may comprise CD25 or an IL-2 binding fragment thereof, such as human CD25 or a human IL-2 (hIL-2) binding fragment thereof. Exemplary hIL-2 binding fragments of human CD25 include residues 22-240 (SEQ ID NO: 11) and residues 22-223 (SEQ ID NO: 12) and residues 22-186 (SEQ ID NO: 14) of full-length hCD25 (SEQ ID NO: 10).

In some embodiments the PD-1 binding moiety is nivolumab or an antigen binding fragment thereof, and the CD25 moiety is an IL-2 binding fragment of hCD25, such as hCD25 variant a (SEQ ID NO: 11), hCD25 variant b (SEQ ID NO: 12) or hCD25 variant d (SEQ ID NO: 14).

In some embodiments the CD25 moiety, such as hCD25 variant a, hCD25 variant b, or hCD25 variant d, is fused to the C-terminus of one of the heavy chains of an anti-PD1 antibody, such as nivolumab. In some embodiments the CD25 moiety, such as hCD25 variant a or hCD25 variant b, is fused to the C-termini of both of the heavy chains of an anti-PD1 antibody, such as nivolumab. In some embodiments the antibody heavy chain is linked to the CD25 moiety via a linker, such as (G₄S)₃ (SEQ ID NO: 7).

Exemplary mouse reagent constructs of the present invention comprise one CD25-4H2 heavy chain fusion polypeptide comprising the sequence of SEQ ID NO: 8 or 9, one 4H2 heavy chain comprising the sequence of SEQ ID NO: 5, and two 4H2 light chains comprising the sequence of SEQ ID NO: 6. Other exemplary constructs of the present invention comprise two CD25-4H2 heavy chain fusion polypeptides comprising the sequence of SEQ ID NO: 8 and two 4H2 light chains comprising the sequence of SEQ ID NO: 6; or alternatively two CD25-4H2 heavy chain fusion polypeptides comprising the sequence of SEQ ID NO: 9 and two 4H2 light chains comprising the sequence of SEQ ID NO: 6.

Exemplary human therapeutic constructs of the present invention comprise one CD25-nivolumab heavy chain fusion polypeptide comprising the sequence of SEQ ID NO: 28, one nivolumab heavy chain comprising the sequence of SEQ ID NO: 25 or 26, and two nivolumab light chains comprising the sequence of SEQ ID NO: 27; or alternatively one CD25-nivolumab heavy chain fusion polypeptide comprising the sequence of SEQ ID NO: 29, one nivolumab heavy chain comprising the sequence of SEQ ID NO: 25 or 26, and two nivolumab light chains comprising the sequence of SEQ ID NO: 27; or alternatively one CD25-nivolumab heavy chain fusion polypeptide comprising the sequence of SEQ ID NO: 30, one nivolumab heavy chain comprising the sequence of SEQ ID NO: 25 or 26, and two nivolumab light chains comprising the sequence of SEQ ID NO: 27.

Other exemplary constructs of the present invention comprise two CD25-nivolumab heavy chain fusion polypeptides comprising the sequence of SEQ ID NO: 28 and two nivolumab light chains comprising the sequence of SEQ ID NO: 27; or alternatively two CD25-nivolumab heavy chain fusion polypeptides comprising the sequence of SEQ ID NO: 29 and two nivolumab light chains comprising the sequence of SEQ ID NO: 27; or alternatively two CD25-nivolumab heavy chain fusion polypeptides comprising the sequence of SEQ ID NO: 30 and two nivolumab light chains comprising the sequence of SEQ ID NO: 27.

Additional exemplary therapeutic constructs of the present invention comprise one CD25-pembrolizumab heavy chain fusion polypeptide comprising the sequence of SEQ ID NO: 47, one pembrolizumab heavy chain comprising the sequence of SEQ ID NO: 44 or 45, and two pembrolizumab light chains comprising the sequence of SEQ ID NO: 46; or alternatively one CD25-pembrolizumab heavy chain fusion polypeptide comprising the sequence of SEQ ID NO: 48, one pembrolizumab heavy chain comprising the sequence of SEQ ID NO: 44 or 45, and two pembrolizumab light chains comprising the sequence of SEQ ID NO: 46; or alternatively one CD25-pembrolizumab heavy chain fusion polypeptide comprising the sequence of SEQ ID NO: 49, one pembrolizumab heavy chain comprising the sequence of SEQ ID NO: 44 or 45, and two pembrolizumab light chains comprising the sequence of SEQ ID NO: 46.

Other exemplary constructs of the present invention comprise two CD25-pembrolizumab heavy chain fusion polypeptides comprising the sequence of SEQ ID NO: 47 and two pembrolizumab light chains comprising the sequence of SEQ ID NO: 46; or alternatively two CD25-pembrolizumab heavy chain fusion polypeptides comprising the sequence of SEQ ID NO: 48 and two pembrolizumab light chains comprising the sequence of SEQ ID NO: 46; or alternatively two CD25-pembrolizumab heavy chain fusion polypeptides comprising the sequence of SEQ ID NO: 49 and two pembrolizumab light chains comprising the sequence of SEQ ID NO: 46.

In some embodiments comprising one heavy chain CD25 fusion polypeptide and one heavy chain lacking CD25, the heavy chains are modified by the knob-into-holes approach to promote formation of antibody constructs comprising one of each heavy chain sequence.

The invention also provides nucleic acids encoding the targeting moiety-CD25 moiety polypeptide construct, such as anti-PD-1 CD25 fusion construct, of the present invention, as well as expression vectors comprising these nucleic acids, host cells comprising the vectors, and method of producing the anti-PD-1 CD25 fusion constructs of the present invention by growing the host cells under conditions that allow their production. In some embodiments comprising a targeting moiety that is an antibody, such as an anti-PD-1 antibody, or antigen binding fragment thereof, the heavy and light chain sequences of the antibody are encoded in the same nucleic acid molecule, whereas in other embodiments the heavy and light chains are encoded by separate nucleic acid molecules.

The invention also provides pharmaceutical compositions of the polypeptide constructs of the present invention for use in treating human disease, such as cancer, which compositions comprise salt, buffer and other pharmaceutically acceptable excipients.

The invention further provides compositions of these therapeutic constructs for use in treating human disease, such as cancer, and methods of treating such diseases using the constructs. In various embodiments, the invention provides constructs for, and methods of, treating NSCLC, liver cancer, breast cancer, colorectal cancer (CRC), metastatic melanoma, colon cancer, and/or melanoma. In selected embodiments, the methods of treating cancer comprise constructs for, and methods of, treating NSCLC, liver cancer, and/or breast cancer. In a specific embodiment, the methods of treating cancer comprise constructs for, and methods of, treating NSCLC.

In some embodiments, the polypeptide constructs or anti-PD-1 CD25 fusion constructs of the present invention are administered without administration of IL-2 or any IL-2 derived therapeutic agent. In other embodiments, the polypeptide constructs or anti-PD-1 CD25 fusion constructs of the present invention are administered in combination therapy with human IL-2, or a therapeutically effective derivative thereof, such as aldesleukin (non-glycosylated Al A C125S human IL-2). In further embodiments, the anti-PD-1 CD25 fusion constructs of the present invention are pre-mixed with IL-2 or an IL-2 derived therapeutic agent and the mixture is administered to the subject.

The invention further provides methods of treatment of diseases, such as cancers, in which tumor samples from human patients are screened for their level of IL-2 and a therapeutic construct of the present invention is administered only to patients whose samples show a required minimum level of IL-2.

In other embodiments, the invention further provides methods of treatment of diseases, such as cancers, in which tumor infiltrating lymphocytes (TIL) from human patients are screened for the level of PD-1 expression, and a therapeutic construct of the present invention is administered only to patients whose samples show a required minimum threshold level of PD-1 expression in TIL.

### BRIEF DESCRIPTION OF THE DRAWINGS

FIGS. 1A and 1B are schematic illustrations of two embodiments of the construct of the present invention. FIG. 1A shows an anti-PD1 antibody with a CD25 moiety fused to the C-terminus of one heavy chain, whereas FIG. 1B shows an anti-PD1 antibody with a CD25 moiety fused to the C-terminus of both heavy chains. Heavy and light chain variable domains are shown in gray, constant domains are in white, and CD25 moieties are in black.

FIGS. 2A, 2B and 2C are representations of the IL-2 binding domains of various mCD25 truncation constructs. FIG. 2A provides a representation of a crystal structure of human CD25 with ribbon structures in the sushi 1 and sushi 2 domains (separated by a dashed line) and helices, corresponding roughly to residues 22-182 of SEQ ID NO: 1. Stauber et al. (2006) Proc. Nat'l Acad. Sci. (USA) 103: 2793; PDB 2ERJ. FIG. 2B provides a two-dimensional topographic representation of the primary sequence of the sushi 1 and sushi 2 structural domains of CD25, with the sequence elements contributing to the sushi 2 domain above the dashed line and sequence elements contributing to sequence of the sushi 1 domain below the line. Ribbon structures are represented as arrows drawn N-terminal to C-terminal (as is conventional), and unstructured region of the sequence is represented by a curved dashed line. FIG. 2C provides a lineup of mouse and human CD25 sushi domain sequences, SEQ ID NOs: 11 and 2, respectively. Structurally defined sushi 1 domain sequences are shown in solid boxes, and sushi 2 domain sequences are shown in dashed boxes.

FIGS. 3A and 3B provide sequences for various CD25 truncations of the present invention. FIG. 3A shows mouse CD25 variants a, b and c. FIG. 3B shows human CD25 variants a, b, c, d, e and f. In all cases sushi 2 domain residues are underlined, and structurally defined residues in the sushi 1 domain residues are italicized. In hCD25 variant a in FIG. 3B residues in human CD25 found in beta ribbons are in bold.

FIG. 4 provides surface plasmon resonance binding data for the three constructs illustrated in FIG. 3A to mIL-2. See Example 1. SPR signal is provided (in nm), from left to right, as the sensor chip is flowed with mIL-2 for baseline; flowed with only buffer as a wash; flowed with a fusion construct of an anti-mPD1 antibody (4H2) to one of the three mCD25 truncations to load the surface; flowed with buffer; flowed with mIL-2 for association; and flowed with buffer only for dissociation. The abscissa is a timeline from 0 to 240 minutes, and the ordinate is a linear scale from 0 to 1.2 nm. The lower (A), middle (B) and upper (C) traces are for the mCD25 truncations from variants a, b and c from FIG. 3A, respectively. Variant c, comprising only sushi 1 domain sequence, does not bind to mIL-2, whereas the variants a and b, which comprise both sushi 1 and sushi 2 domain sequences, and varying additional residues at the carboxy termini, do.

FIGS. 5A and 5B provide sequences for mCD25 anti-mPD1 mAb fusion constructs of the present invention. FIG. 5A (SEQ ID NO: 8) provides the heavy chain of anti-mPD-1 mAb 4H2 (SEQ ID NO: 5) linked to mCD25 variant a (italic, SEQ ID NO: 2) by a (G₄S)₃ linker (double underlined, SEQ ID NO: 7). FIG. 5B (SEQ ID NO: 9) provides the heavy chain of anti-mPD-1 mAb 4H2 (SEQ ID NO: 5) linked to mCD25 variant b (italic, SEQ ID NO: 3) by a (G₄S)₃ linker (double underlined, SEQ ID NO: 7).

FIGS. 6A, 6B and 6C provide sequences for hCD25 anti-hPD1 (nivolumab) mAb fusion constructs of the present invention. FIG. 6A (SEQ ID NO: 28) provides the heavy chain of anti-hPD-1 mAb nivolumab (SEQ ID NO: 26) linked to hCD25 variant a (italic, SEQ ID NO: 11) by a (G₄S)₃ linker (double underlined, SEQ ID NO: 7). FIG. 6B (SEQ ID NO: 29) provides the heavy chain of anti-hPD-1 mAb nivolumab (SEQ ID NO: 26) linked to hCD25 variant b (italic, SEQ ID NO: 12) by a (G₄S)₃ linker (double underlined, SEQ ID NO: 7). FIG. 6C (SEQ ID NO: 30) provides the heavy chain of anti-hPD-1 mAb nivolumab (SEQ ID NO: 26) linked to hCD25 variant d (italic, SEQ ID NO: 14) by a (G₄S)₃ linker (double underlined, SEQ ID NO: 7). The heavy chain variable domains in FIGS. 6A-6C are underlined, and CDRs are bolded. Analogous pembrolizumab constructs are provided at SEQ ID NOs: 47, 48 and 49.

FIGS. 7A, 7B and 7C are variants of the sequences of FIGS. 6A, 6B and 6C, respectively, except that the nivolumab hIgG4 S228P heavy chain constant domain is replaced with the effectorless hIgG1.3. The nivolumab heavy chain with hIgG1.3 instead of hIgG4 S228P is provided at SEQ ID NOs: 31 and 32. The sequences provided at FIGS. 7A, 7B and 7C are provided at SEQ ID NOs: 33, 34 and 35, respectively. The heavy chain variable domains in FIGS. 7A-7C are underlined, and CDRs are bolded. Analogous pembrolizumab hIgG1.3 constructs are provided at SEQ ID NOs: 52, 53 and 54.

FIGS. 8A-8D provide data characterizing cell lines engineered to illustrate the effects of the constructs of the present invention. See Example 2. FIG. 8A shows sorting of HEK-Blue™ IL-2 cells, which express all three subunits of IL-2 receptor, after deletion of hCD25, showing a substantial population of hCD25⁻ cells. FIG. 8B shows sorting cells from the sort of FIG. 8A confirming that they remain CD122 (IL-2Rβ) and CD132 (IL-2Rγ) positive. The CD25− HEK-Blue™ cells from FIG. 8A were then transduced with mPD-1 or hPD-1 and sorted. FIGS. 8C and 8D show that these cell populations are both hCD25− and mPD-1+ and hPD-1+, respectively. These cells find use in testing the anti-PD-1−hCD25 fusion constructs of the present invention, in which the anti-PD-1 moiety may be an anti-mPD-1 antibody (e.g. mAb 4H2) or an anti-hPD-1 antibody (e.g. nivolumab).

FIG. 9 shows a titration of mIL-2 binding to CD25+ (upper curve) and CD25− (lower curve) HEK-Blue™ IL-2 cells, confirming the importance of CD25 for IL-2 binding and signaling. See Example 3. Signaling data are reported as ABS 620 nM in an alkaline phosphatase activity assay based on differential expression of the SEAP (secreted embryonic alkaline phosphatase) reporter gene in the HEK-Blue™ reporter cell line.

FIGS. 10A and 10B show titrations of mIL-2 signaling in CD25−mPD-1+ HEK-Blue™ IL-2 cells in the presence of a hemi-mCD25 modified (4H2 mG1 D265A KK CD25.b+4H2 mG1 D265A blank) and a fully mCD25 modified ((4H2 mG1 D265A KK CD25.b)₂) anti-mPD-1 antibody (4H2), respectively. See Example 3. The hemi- and fully modified constructs showed similar ability to enhance mIL-2 signaling in a dose responsive manner. FIG. 10C presents data essentially replicating those in FIG. 10B, but also including control experiments with an anti-KLH mAb (mAb 29D6) fusion to CD25, demonstrating that the observed effects depend on PD-1 binding. For FIG. 10A, mIL-2 only is lowest curve; 28 pM fusion is next higher curve; 280 pM fusion is next higher curve; 2.8 nM fusion is upper curve. For FIG. 10B, mIL-2 only is lowest curve; 26 pM fusion is next higher curve; 260 pM fusion is next higher curve; 2.8 nM fusion is upper curve. For FIG. 10C, 2.6 nM anti-mPD-1 fusion is the uppermost curve; 260 pM anti-mPD-1 fusion is the next lower curve; 26 pM anti-mPD-1 fusion is the next lower curve; the lower curve comprises data for 2.6 nM, 260 pM and 26 pM anti-KLH fusion and no fusion.

FIG. 11A shows STAT5 phosphorylation as a function of mIL-2 for primary mouse CD4⁺CD25⁺ (upper curve) and CD8⁺CD25⁻ (lower curve) splenocytes, illustrating the dramatic deficiency of CD25⁻ cells in IL-2 mediated signaling. See Example 4. CD4+ primary T cells and CD8+ primary T cells were gated for PD-1 expression, and then for low CD25 expression. FIGS. 11B and 11C show STAT5 signaling in these two cell preparations, CD8+CD25−PD1^{low} and CD4+CD25−PD1^{med} respectively, when treated with a mixture of mIL-2 and an anti-mPD-1−CD25 fusion construct of the present invention. For FIG. 11B, 4H2-mCD25.b+mIL-2 is the upper curve at 25 nM; IL-2 only is the second highest curve at nM; KLH-mCD25.b+mIL-2 is the third highest curve at 25 nM; KLH-mCD25.b is the fourth highest (nearly baseline) curve at 25 nM; and 4H2-mCD25.b is the lowest curve (essentially at baseline throughout). For FIG. 11C, 4H2-mCD25.b+mIL-2 is upper curve at 25 nM; KLH-mCD25.b+mIL-2 is the second highest curve at 25 nM; IL-2 only is third highest curve at 25 nM; KLH-mCD25.b is the fourth highest (nearly baseline) curve at 25 nM; and 4H2-mCD25.b is the lowest curve (essentially at baseline throughout).

FIGS. 12A-12C show plots of single cell RNA sequencing data from tumor infiltrated lymphocytes (TIL). Data are presented for in 9,055 single T cells from 14 NSCLC patients. The dimensional reduction analysis (t-SNE) projections show sixteen main clusters, including seven for CD8+ T cells, seven for conventional CD4+ T cells and two for regulatory T cells. Each dot corresponds to a single cell, with darker color representing more intense staining. Gene Expression Omnibus Accession No: GSE99254; Guo et al. (2018) Nat. Med. 24:978. FIG. 12A shows expression of IL-2, IL-15, IL2RA, IL2RB, IL2RG and IL15RA, as indicated. FIG. 12B shows expression of PDCD1, KLRC1, CD8A, FCRL8, CRTAM and LAG3, as indicated. FIG. 12C shows expression of FOXP3, CCR8 and CTLA-4, as indicated. See Example 5. Comparison of FIG. 12A with FIG. 12B shows that cells that express PDCD1, KLRC1, CD8A, FCRL8, CRTAM and LAG3 tend to also express IL2RB and IL2RG. Comparison of FIG. 12A with FIG. 12C shows that cells that express PDCD1, KLRC1, CD8A, FCRL8, CRTAM and LAG3 tend not to express T_{reg} markers FOXP3, CCR8 and CTLA-4.

### DETAILED DESCRIPTION OF THE INVENTION

### Definitions

In order that the present disclosure may be more readily understood, certain terms are first defined. As used in this application, except as otherwise expressly provided herein, each of the following terms shall have the meaning set forth below. Additional definitions are set forth throughout the application.

“Administering” refers to the physical introduction of a composition comprising a therapeutic agent to a subject, using any of the various methods and delivery systems known to those skilled in the art. Preferred routes of administration for antibodies of the invention include intravenous, intraperitoneal, intramuscular, subcutaneous, spinal or other parenteral routes of administration, for example by injection or infusion. The phrase “parenteral administration” as used herein means modes of administration other than enteral and topical administration, usually by injection, and includes, without limitation, intravenous, intraperitoneal, intramuscular, intraarterial, intrathecal, intralymphatic, intralesional, intracapsular, intraorbital, intracardiac, intradermal, transtracheal, subcutaneous, subcuticular, intraarticular, subcapsular, subarachnoid, intraspinal, epidural and intrasternal injection and infusion, as well as in vivo electroporation. Alternatively, an antibody of the invention can be administered via a non-parenteral route, such as a topical, epidermal or mucosal route of administration, for example, intranasally, orally, vaginally, rectally, sublingually or topically. Administering can also be performed, for example, once, a plurality of times, and/or over one or more extended periods. Administration may be performed by one or more individual, including but not limited to, a doctor, a nurse, another healthcare provider, or the patient himself or herself.

An “antibody” (Ab) shall include, without limitation, a glycoprotein immunoglobulin which binds specifically to an antigen and comprises at least two heavy (H) chains and two light (L) chains interconnected by disulfide bonds, or an antigen-binding portion thereof. Each H chain comprises a heavy chain variable region (abbreviated herein as V_{H}) and a heavy chain constant region. The heavy chain constant region comprises three domains, C_{H1}, C_{H2} and C_{H3}. Each light chain comprises a light chain variable region (abbreviated herein as V_{L}) and a light chain constant region. The light chain constant region is comprised of one domain, C_{L}. The V_{H} and V_{L} regions can be further subdivided into regions of hypervariability, termed complementarity determining regions (CDRs), interspersed with regions that are more conserved, termed framework regions (FR). Each V_{H} and V_{L} is composed of three CDRs and four FRs, arranged from amino-terminus to carboxy-terminus in the following order: FR1, CDR1, FR2, CDR2, FR3, CDR3, FR4. The variable regions of the heavy and light chains contain a binding domain that interacts with an antigen.

As used herein, and in accord with conventional interpretation, an antibody that is described as comprising “a” heavy chain and/or “a” light chain refers to antibodies that comprise “at least one” of the recited heavy and/or light chains, and thus will encompass antibodies having two or more heavy and/or light chains. Specifically, antibodies so described will encompass conventional antibodies having two substantially identical heavy chains and two substantially identical light chains. Antibody chains may be substantially identical but not entirely identical if they differ due to post-translational modifications, such as C-terminal cleavage of lysine residues, alternative glycosylation patterns, etc.

Unless indicated otherwise or clear from the context, an antibody defined by its target specificity (e.g. an “anti-PD-1 antibody”) refers to antibodies that can bind to its human target (e.g. human PD-1). Such antibodies may or may not bind to PD-1 from other species.

The immunoglobulin may derive from any of the commonly known isotypes, including but not limited to IgA, secretory IgA, IgG and IgM. The IgG isotype may be divided in subclasses in certain species: IgG1, IgG2, IgG3 and IgG4 in humans, and IgG1, IgG2a, IgG2b and IgG3 in mice. IgG antibodies may be referred to herein by the symbol gamma (γ) or simply “G,” e.g. IgG1 may be expressed as “γ1” or as “G1,” as will be clear from the context. “Isotype” refers to the antibody class (e.g., IgM or IgG1) that is encoded by the heavy chain constant region genes. “Antibody” includes, by way of example, both naturally occurring and non-naturally occurring antibodies; monoclonal and polyclonal antibodies; chimeric and humanized antibodies; human or nonhuman antibodies; wholly synthetic antibodies; and single chain antibodies. Unless otherwise indicated, or clear from the context, antibodies disclosed herein are human IgG1 antibodies.

An “isolated antibody” refers to an antibody that is substantially free of other antibodies having different antigenic specificities (e.g., an isolated antibody that binds specifically to PD-1 is substantially free of antibodies that bind specifically to antigens other than PD-1). An isolated antibody that binds specifically to PD-1 may, however, cross-react with other antigens, such as PD-1 molecules from different species. Moreover, an isolated antibody may be substantially free of other cellular material and/or chemicals. By comparison, an “isolated” nucleic acid refers to a nucleic acid composition of matter that is markedly different, i.e., has a distinctive chemical identity, nature and utility, from nucleic acids as they exist in nature. For example, an isolated DNA, unlike native DNA, is a free-standing portion of a native DNA and not an integral part of a larger structural complex, the chromosome, found in nature. Further, an isolated DNA, unlike native DNA, can be used as a PCR primer or a hybridization probe for, among other things, measuring gene expression and detecting biomarker genes or mutations for diagnosing disease or predicting the efficacy of a therapeutic. An isolated nucleic acid may also be purified so as to be substantially free of other cellular components or other contaminants, e.g., other cellular nucleic acids or proteins, using standard techniques well known in the art.

The term “monoclonal antibody” (“mAb”) refers to a preparation of antibody molecules of single molecular composition, i.e., antibody molecules whose primary sequences are essentially identical, and which exhibits a single binding specificity and affinity for a particular epitope. Monoclonal antibodies may be produced by hybridoma, recombinant, transgenic or other techniques known to those skilled in the art.

The term “afucosylated,” as used herein, refers to individual antibody heavy chains in which the N-linked glycan contains no fucose residues. The term “nonfucosylated” as used herein, refers to a preparation of antibodies containing antibodies with afucosylated heavy chains, and unless otherwise indicated over 95% afucosylated heavy chains. Such preparations of antibodies may be used as therapeutic compositions.

A “human” antibody (HuMAb) refers to an antibody having variable regions in which both the framework and CDR regions are derived from human germline immunoglobulin sequences. Furthermore, if the antibody contains a constant region, the constant region also is derived from human germline immunoglobulin sequences. The human antibodies of the invention may include amino acid residues not encoded by human germline immunoglobulin sequences (e.g., mutations introduced by random or site-specific mutagenesis in vitro or by somatic mutation in vivo). However, the term “human antibody”, as used herein, is not intended to include antibodies in which CDR sequences derived from the germline of another mammalian species, such as a mouse, have been grafted onto human framework sequences. The terms “human” antibodies and “fully human” antibodies and are used synonymously.

An “antibody fragment” refers to a portion of a whole antibody, generally including the “antigen-binding portion” (“antigen-binding fragment”) of an intact antibody which retains the ability to bind specifically to the antigen bound by the intact antibody, or the Fc region of an antibody which retains FcR binding capability. Exemplary antibody fragments include Fab fragments and single chain variable domain (scFv) fragments.

“Antibody-dependent cell-mediated cytotoxicity” (“ADCC”) refers to an in vitro or in vivo cell-mediated reaction in which nonspecific cytotoxic cells that express FcRs (e.g., natural killer (NK) cells, macrophages, neutrophils and eosinophils) recognize antibody bound to a surface antigen on a target cell and subsequently cause lysis of the target cell. In principle, any effector cell with an activating FcR can be triggered to mediate ADCC.

“Cancer” refers a broad group of various diseases characterized by the uncontrolled growth of abnormal cells in the body. Unregulated cell division and growth divide and grow results in the formation of malignant tumors or cells that invade neighboring tissues and may also metastasize to distant parts of the body through the lymphatic system or bloodstream.

A “cell surface receptor” refers to molecules and complexes of molecules capable of receiving a signal and transmitting such a signal across the plasma membrane of a cell.

An “effector cell” refers to a cell of the immune system that expresses one or more FcRs and mediates one or more effector functions. Preferably, the cell expresses at least one type of an activating Fc receptor, such as, for example, human FcγRIII, and performs ADCC effector function. Examples of human leukocytes which mediate ADCC include peripheral blood mononuclear cells (PBMCs), NK cells, monocytes, macrophages, neutrophils and eosinophils.

“Effector function” refers to the interaction of an antibody Fc region with an Fc receptor or ligand, or a biochemical event that results therefrom. Exemplary “effector functions” include Clq binding, complement dependent cytotoxicity (CDC), Fc receptor binding, FcγR-mediated effector functions such as ADCC and antibody dependent cell-mediated phagocytosis (ADCP), and down-regulation of a cell surface receptor (e.g., the B cell receptor; BCR). Such effector functions generally require the Fc region to be combined with a binding domain (e.g., an antibody variable domain).

An “Fc receptor” or “FcR” is a receptor that binds to the Fc region of an immunoglobulin. FcRs that bind to an IgG antibody comprise receptors of the FcγR family, including allelic variants and alternatively spliced forms of these receptors. The FcγR family consists of three activating (FcγRI, FcγRIII, and FcγRIV in mice; FcγRIA, FcγRIIA, and FcγRIIIA in humans) receptors and one inhibitory (FcγRIIB) receptor. Various properties of human FcγRs are summarized in Table 1. The majority of innate effector cell types co-express one or more activating FcγR and the inhibitory FcγRIIB, whereas natural killer (NK) cells selectively express one activating Fc receptor (FcγRIII in mice and FcγRIIIA in humans) but not the inhibitory FcγRIIB in mice and humans.

An “Fc region” (fragment crystallizable region) or “Fc domain” or “Fc” refers to the C-terminal region of the heavy chain of an antibody that mediates the binding of the immunoglobulin to host tissues or factors, including binding to Fc receptors located on various cells of the immune system (e.g., effector cells) or to the first component (C1q) of the classical complement system. Thus, the Fc region is a polypeptide comprising the constant region of an antibody excluding the first constant region immunoglobulin domain. In IgG, IgA and IgD antibody isotypes, the Fc region is composed of two identical protein fragments, derived from the second (C_{H2}) and third (C_{H3}) constant domains of the antibody's two heavy chains; IgM and IgE Fc regions contain three heavy chain constant domains (C_{H} domains 2-4) in each polypeptide chain. For IgG, the Fc region comprises immunoglobulin domains Cγ2 and Cγ3 and the hinge between Cγ1 and Cγ2. Although the boundaries of the Fc region of an immunoglobulin heavy chain might vary, the human IgG heavy chain Fc region is usually defined to stretch from an amino acid residue at position C226 or P230 to the carboxy-terminus of the heavy chain, wherein the numbering is according to the EU index as in Kabat. The C_{H2} domain of a human IgG Fc region extends from about amino acid 231 to about amino acid 340, whereas the C_{H3} domain is positioned on C-terminal side of a C_{H2} domain in an Fc region, i.e., it extends from about amino acid 341 to about amino acid 447 of an IgG. As used herein, the Fc region may be a native sequence Fc or a variant Fc. Fc may also refer to this region in isolation or in the context of an Fc-comprising protein polypeptide such as a “binding protein comprising an Fc region,” also referred to as an “Fe fusion protein” (e.g., an antibody or immunoadhesin).

TABLE 1
Properties of Human FcγRs

| | Allelic | Affinity for | Isotype | |
|---|---|---|---|---|
| Fcγ | variants | human IgG | preference | Cellular distribution |
| FcγRI | None | High | IgG1 = | Monocytes, |
| | described | (K_{D}~10 | 3 > 4 >> 2 | macrophages, |
| | | nM) | | activated neutrophils, |
| | | | | dendritic cells? |
| FcγRIIA | H131 | Low to | IgG1 > | Neutrophils, |
| | | medium | 3 > 2 > 4 | monocytes, macro- |
| | R131 | Low | IgG1 > 3 > | phages, eosinophils, |
| | | | 4 > 2 | dendritic cells, |
| | | | | platelets |
| FcγRIIIA | V158 | Medium | IgG1 = | NK cells, monocytes, |
| | | | 3 >> 4 > 2 | macrophages, mast |
| | F158 | Low | IgG1 = | cells, eosinophils, |
| | | | 3 >> 4 > 2 | dendritic cells? |
| FcγRIIB | I232 | Low | IgG1 = | B cells, monocytes, |
| | | | 3 = 4 > 2 | macrophages, |
| | T232 | Low | IgG1 = | dendritic cells, |
| | | | 3 = 4 > 2 | mast cells |

An “immune response” refers to a biological response within a vertebrate against foreign agents, which response protects the organism against these agents and diseases caused by them. The immune response is mediated by the action of a cell of the immune system (for example, a T lymphocyte, B lymphocyte, natural killer (NK) cell, macrophage, eosinophil, mast cell, dendritic cell or neutrophil) and soluble macromolecules produced by any of these cells or the liver (including antibodies, cytokines, and complement) that results in selective targeting, binding to, damage to, destruction of, and/or elimination from the vertebrate's body of invading pathogens, cells or tissues infected with pathogens, cancerous or other abnormal cells, or, in cases of autoimmunity or pathological inflammation, normal human cells or tissues.

An “immunomodulator” or “immunoregulator” refers to a component of a signaling pathway that may be involved in modulating, regulating, or modifying an immune response. “Modulating,” “regulating,” or “modifying” an immune response refers to any alteration in a cell of the immune system or in the activity of such cell. Such modulation includes stimulation or suppression of the immune system which may be manifested by an increase or decrease in the number of various cell types, an increase or decrease in the activity of these cells, or any other changes which can occur within the immune system. Both inhibitory and stimulatory immunomodulators have been identified, some of which may have enhanced function in a tumor microenvironment. In preferred embodiments of the disclosed invention, the immunomodulator is located on the surface of a T cell. An “immunomodulatory target” or “immunoregulatory target” is an immunomodulator that is targeted for binding by, and whose activity is altered by the binding of, a substance, agent, moiety, compound or molecule. Immunomodulatory targets include, for example, receptors on the surface of a cell (“immunomodulatory receptors”) and receptor ligands (“immunomodulatory ligands”).

“Immunotherapy” refers to the treatment of a subject afflicted with, or at risk of contracting or suffering a recurrence of, a disease by a method comprising inducing, enhancing, suppressing or otherwise modifying an immune response.

“PD-1 Moiety,” as used herein, refers to the PD-1 binding component of the bispecific construct of the present invention. Unless otherwise indicated, or clear from the context, PD-1 as used herein refers to human PD-1 (hPD-1), and anti-PD-1 antibody refers to an anti-hPD-1 antibody. The PD-1 binding component may be the antigen binding site of an anti-PD-1 antibody, such as anti-mPD-1 mAb 4H2, or anti-hPD-1 mAb nivolumab or pembrolizumab. Anti-mPD-1 mAb 4H2 is described at Li et al. (2009) Clin. Cancer Res. 15: 1623. Nivolumab is described, e.g., in U.S. Pat. Nos. 8,008,449 and 8,779,105, and also in WO 2013/173223. Pembrolizumab is described, e.g., in U.S. Pat. No. 8,354,509. Sequences for these antibodies are also provided in the Sequence Listing.

“CD25 Moiety,” as used herein, refers to an IL-2-binding polypeptide that comprises some or all of the sequence of CD25 (IL-2Rα), such as mouse CD25 (mCD25) or human CD25 (hCD25). Unless otherwise indicated, or clear from the context, CD25 as used herein refers to human CD25. CD25 is the alpha subunit of the IL-2 receptor (IL-2R), along with CD122 (IL-2Rβ) and CD132 (IL-2Rγ). A CD25 Moiety will typically comprise a full-length CD25 sequence or a truncation that retains IL-2 binding activity. Exemplary mouse and human CD25 truncations include those provided at SEQ ID NOs: 2 and 3, and SEQ ID NOs: 11, 12 and 14, respectively.

A “polypeptide construct,” as used herein with reference to the compositions of matter of the present invention, refers to a bispecific construct comprising a targeting moiety, such as PD-1 binding moiety, and a CD25 moiety. Such constructs may comprise one or more than one of each of the moieties, such as two PD-1 moieties and one CD25 moiety or two PD-1 moieties and two CD25 moieties. Such polypeptide constructs may synonymously be referred to as anti-PD-1 CD25 fusion constructs. Such polypeptide constructs may comprise one or more polypeptide chains, including two or more polypeptide chains comprising different sequences (e.g. antibody heavy and light chains), such as antibodies comprising one or more antibody light chains and one or more fusion constructs comprising an antibody heavy chain fused to a CD25 moiety, such as an antibody comprising two light chains and two heavy chain-CD25 fusion polypeptides.

“Hemi-CD25 modified,” as used herein, refers to a bivalent antibody comprising two heavy chains in which only one of the two heavy chains further comprises a CD25 moiety. It is as opposed to a “fully CD25 modified” construct, in which both heavy chains are modified to further comprise a CD25 moiety. In hemi-CD25 modified embodiments, the CH3 domains of the hIgG4 antibodies nivolumab and pembrolizumab may be modified using the “knob-into-hole” method of Ridgway et al. (1996) Protein Eng. 9:617, as applied to hIgG4 variants in Spiess et al. (2013) J. Biol. Chem. 288:26583, to generate two separate heavy chain constant domain sequences that preferentially assemble into heterodimers, favoring the formation of hemi-CD25 modified antibodies rather than unmodified or fully CD25 modified species. Analogous knob-into-hole modifications may be made in hIgG1 variants of nivolumab and pembrolizumab, such as hIgG1.3 variants, as described. Ridgway et al. (1996) Protein Eng. 9:617; Merchant et al. (1998) Nat. Biotechnol. 16:677.

“Potentiating an endogenous immune response” means increasing the effectiveness or potency of an existing immune response in a subject. This increase in effectiveness and potency may be achieved, for example, by overcoming mechanisms that suppress the endogenous host immune response or by stimulating mechanisms that enhance the endogenous host immune response.

A “protein” refers to a chain comprising at least two consecutively linked amino acid residues, with no upper limit on the length of the chain. One or more amino acid residues in the protein may contain a modification such as, but not limited to, glycosylation, phosphorylation or disulfide bond formation. The term “protein” is used interchangeable herein with “polypeptide.”

A “subject” includes any human or non-human animal. The term “non-human animal” includes, but is not limited to, vertebrates such as nonhuman primates, sheep, dogs, rabbits, rodents such as mice, rats and guinea pigs, avian species such as chickens, amphibians, and reptiles. In preferred embodiments, the subject is a mammal such as a nonhuman primate, sheep, dog, cat, rabbit, ferret or rodent. In more preferred embodiments of any aspect of the disclosed invention, the subject is a human. The terms, “subject” and “patient” are used interchangeably herein.

“Targeting moiety,” as used herein, refers to the component of the fusion constructs of the present invention that binds to a surface marker on a desired target cell, such as anti-tumor CD8+ effector T cells, and promotes delivery of IL-2 to such target cells by providing CD25 to enhance IL-2 receptor activity. One such targeting moiety is PD-1. Alternative targeting moieties include, for example, NKG2a, CD8a, FcRL6, CRTAM and LAG3. Targeting moieties will typically comprise an antibody, or antigen binding portion thereof, that specifically binds to the alternative target, provided that any antigen binding portion an also be fused to CD25 or an active fragment thereof. Unless clear from the context, all methods and constructs of the present invention reciting anti-PD-1 antibodies also provide alternative embodiments using an alternative targeting moiety in place of anti-PD-1.

A “therapeutically effective amount” or “therapeutically effective dosage” of a drug or therapeutic agent, such as an Fc fusion protein of the invention, is any amount of the drug that, when used alone or in combination with another therapeutic agent, promotes disease regression evidenced by a decrease in severity of disease symptoms, an increase in frequency and duration of disease symptom-free periods, or a prevention of impairment or disability due to the disease affliction. A therapeutically effective amount or dosage of a drug includes a “prophylactically effective amount” or a “prophylactically effective dosage”, which is any amount of the drug that, when administered alone or in combination with another therapeutic agent to a subject at risk of developing a disease or of suffering a recurrence of disease, inhibits the development or recurrence of the disease. The ability of a therapeutic agent to promote disease regression or inhibit the development or recurrence of the disease can be evaluated using a variety of methods known to the skilled practitioner, such as in human subjects during clinical trials, in animal model systems predictive of efficacy in humans, or by assaying the activity of the agent in in vitro assays.

By way of example, an anti-cancer agent promotes cancer regression in a subject. In preferred embodiments, a therapeutically effective amount of the drug promotes cancer regression to the point of eliminating the cancer. “Promoting cancer regression” means that administering an effective amount of the drug, alone or in combination with an anti-neoplastic agent, results in a reduction in tumor growth or size, necrosis of the tumor, a decrease in severity of at least one disease symptom, an increase in frequency and duration of disease symptom-free periods, a prevention of impairment or disability due to the disease affliction, or otherwise amelioration of disease symptoms in the patient. In addition, the terms “effective” and “effectiveness” with regard to a treatment includes both pharmacological effectiveness and physiological safety. Pharmacological effectiveness refers to the ability of the drug to promote cancer regression in the patient. Physiological safety refers to the level of toxicity, or other adverse physiological effects at the cellular, organ and/or organism level (adverse effects) resulting from administration of the drug.

By way of example for the treatment of tumors, a therapeutically effective amount or dosage of the drug preferably inhibits cell growth or tumor growth by at least about 20%, more preferably by at least about 40%, even more preferably by at least about 60%, and still more preferably by at least about 80% relative to untreated subjects. In the most preferred embodiments, a therapeutically effective amount or dosage of the drug completely inhibits cell growth or tumor growth, i.e., preferably inhibits cell growth or tumor growth by 100%. The ability of a compound to inhibit tumor growth can be evaluated in an animal model system, such as the CT26 colon adenocarcinoma, MC38 colon adenocarcinoma and Sa1N fibrosarcoma mouse tumor models described herein, which are predictive of efficacy in human tumors. Alternatively, this property of a composition can be evaluated by examining the ability of the compound to inhibit cell growth, such inhibition can be measured in vitro by assays known to the skilled practitioner. In other preferred embodiments of the invention, tumor regression may be observed and continue for a period of at least about 20 days, more preferably at least about 40 days, or even more preferably at least about 60 days.

“Treatment” or “therapy” of a subject refers to any type of intervention or process performed on, or administering an active agent to, the subject with the objective of reversing, alleviating, ameliorating, inhibiting, slowing down or prevent the onset, progression, development, severity or recurrence of a symptom, complication, condition or biochemical indicia associated with a disease.

### Anti-PD-1 CD25 Fusion Constructs for the Treatment of Cancer

Cytokines like IL-2 are potent activators of immune responses, and find use in treatment of cancers where they enhance anti-tumor immune response. In one aspect, the present invention provides anti-PD-1 CD25 polypeptide fusion constructs for use in treating diseases, such as cancer. Such constructs comprise a PD-1 binding moiety, such as an anti-PD-1 antibody or antigen binding fragment thereof, fused to a CD25 moiety, or IL-2 binding fragment thereof. Such constructs bind to endogenous IL-2 through the CD25 (IL-2Rα) moiety and redirect it to PD-1 expressing cells, such as NK cells and CD8⁺ effector T cells (T _{eff}) expressing CD122 (IL-2Rβ) and CD132 (IL-2Rγ) but not CD25.

In the absence of the anti-PD-1 CD25 fusion constructs of the present invention, immunosuppressive regulatory T cells (T_{reg)}express all three IL-2R subunits (α, β and γ) and bind to IL-2 with high affinity (K_{d}˜10 pm), whereas NK cells and T_{eff} express only the β and γ subunits and bind with intermediate affinity (K_{d}˜1 nM). Spolski et al. (2018) Nat. Rev. Immunol. 18:648. This balance of IL-2 affinities ensures that T_{reg} will outcompete NK cells and T_{eff} for IL-2 stimulation when IL-2 levels are low, thus maintaining an immunosuppressed resting state. But at high levels of IL-2, NK cells and T_{eff} will bind IL-2, driving their growth and expansion an active immune response. By supplying the missing IL-2Rα subunit to PD-1+ T_{eff}, the anti-PD-1 CD25 fusion constructs of the present invention complete the high affinity trimeric IL-2 receptor complex and redirect IL-2 binding away from immunosuppressive T_{eff} and toward anti-tumor T_{eff}. Such redirection of IL-2 promotes anti-tumor responses without systemic administration of potentially toxic exogenous IL-2, while limit the stimulatory effects of IL-2 to PD-1⁺ cell populations.

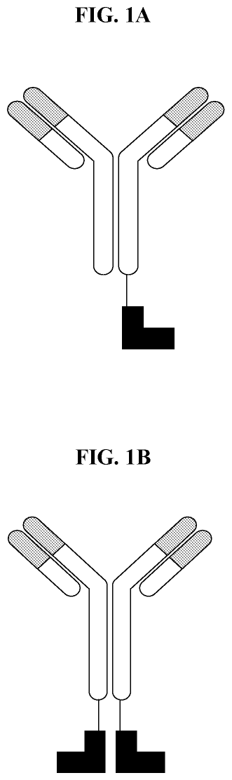

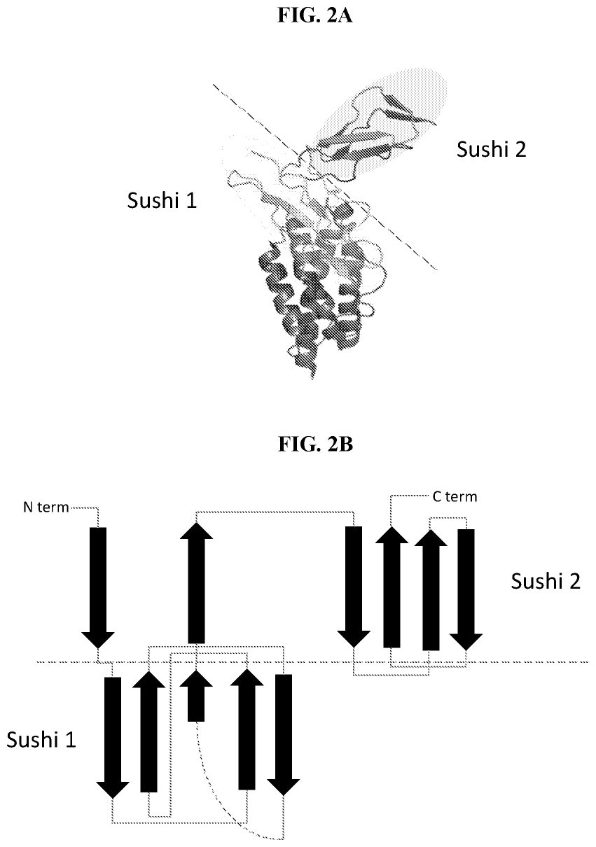

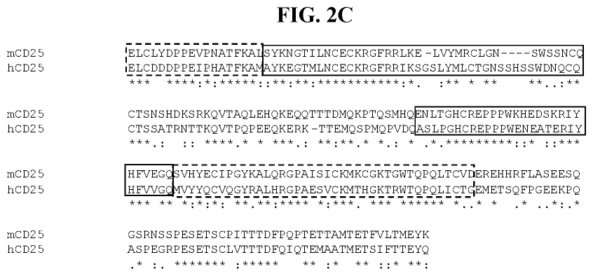

In one embodiment, the PD-1 moiety is and anti-PD-1 antibody and the CD25 moiety is full length extracellular domain of CD25 (referred to herein as full-length CD25) or an IL-2 binding truncation of that sequence. Schematic illustrations of constructs with CD25 bound to the C terminus of one antibody heavy chain, and to the C terminus of both antibody heavy chains, are provided at FIGS. 1A and 1B, respectively. The tertiary, secondary and primary structures of CD25 are schematically illustrated in FIGS. 2A, 2B and 2C, with sushi 2 domains above the line and sushi 1 domains (at the N- and C-termini) below the line in FIGS. 2A and 2B.

The CD25 moiety of the fusion constructs of the present invention may comprise the full extracellular domain of CD25, or a fragment thereof that retain IL-2Rα activity. Such activity is measured by the ability to enhance the binding of IL-2 to cells expressing IL-2Rβ and IL-2Rγ. The sequences of various CD25-related sequences are described at Table 2, and provided in the Sequence Listing (see Table 5). Sequences for CD25 fragments in Table 2 are defined by residue numbering in the full length CD25 sequences provided at SEQ ID NOs: 10 and 1 for human and mouse CD25, respectively.

TABLE 2
CD25 Variant Sequences

| | Human | Mouse |
|---|---|---|
| full-length CD25 | Acc. No. P01589.1 | Acc. No. P01590.1 |
| | SEQ ID NO: 10 (1-272) | SEQ ID NO: 1 (1-268) |
| full-length mature | 22-272 | 22-268 |
| variant a | 22-240 | 22-236 |
| (full-length ECD) | SEQ ID NO: 11 | SEQ ID NO: 2 |
| variant b | 22-223 | 22-219 |
| | SEQ ID NO: 12 | SEQ ID NO: 3 |
| variant c | 40-146 | 40-142 |
| (sushi 1) | SEQ ID NO: 13 | SEQ ID NO: 4 |
| sushi 2 | 22-39 and 147-186 | 22-39 and 143-182 |
| minimal core | 44-85 | 44-81 |
| variant d | 22-186 | |
| | SEQ ID NO: 14 | |
| variant e | 22-145 | |
| | SEQ ID NO: 15 | |
| variant f | 44-85 | |
| | SEQ ID NO: 16 | |

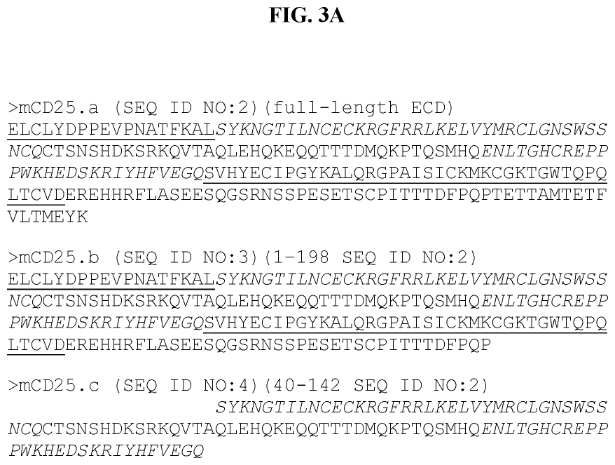

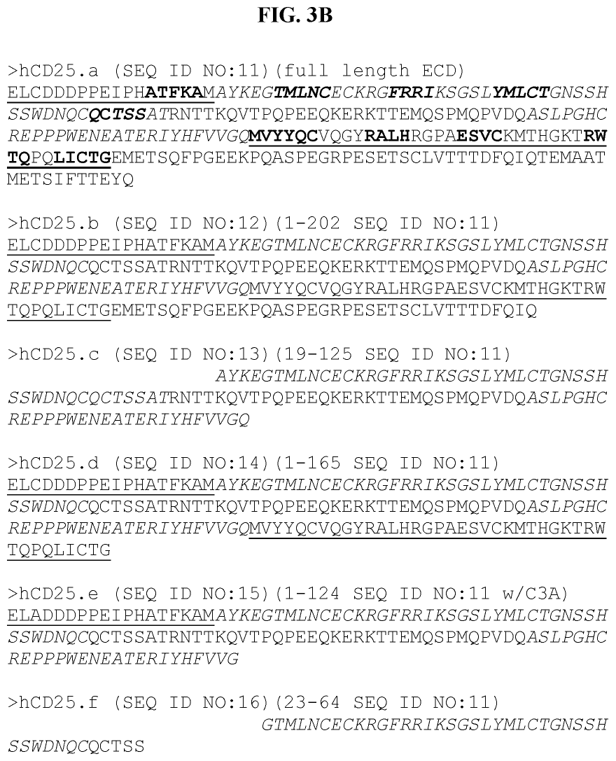

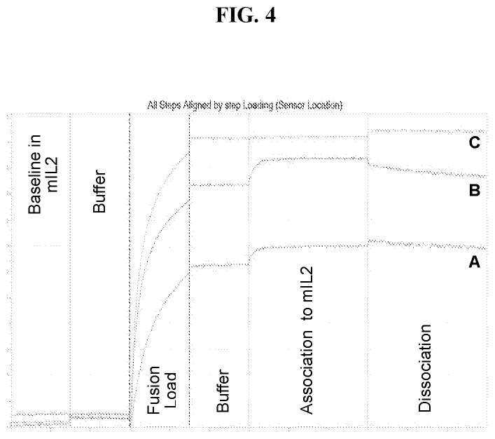

Exemplary mouse CD25 truncations are provided at FIG. 3A, and human counterparts are provided at FIG. 3B. Such truncations may be fused to targeting moieties, such as antibodies to selected targets, such as PD-1. FIG. 4 provides SPR binding data using the mouse CD25 truncations of FIG. 3A, demonstrating that variant a (the full length mCD25 extracellular domain (ECD); SEQ ID NO: 2) binds to mIL-2, variant b (SEQ ID NO: 3) retains full binding affinity (K_{d}=14 nM), but variant c (sushi 1 domain; SEQ ID NO: 4) does not bind.

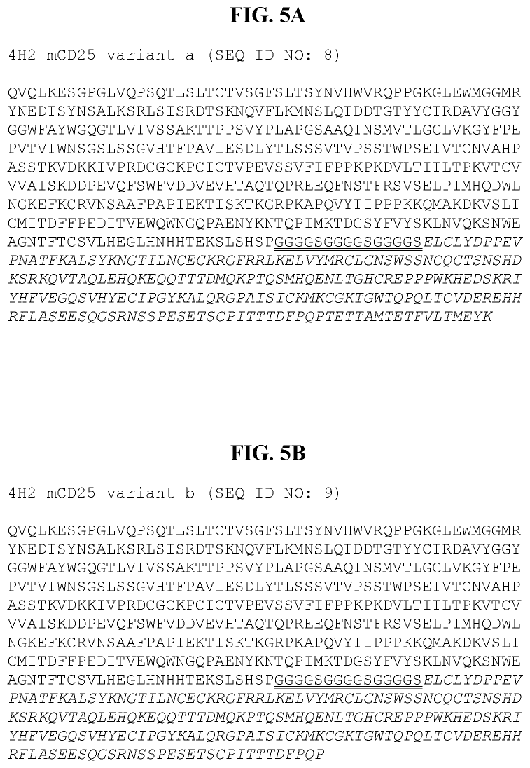

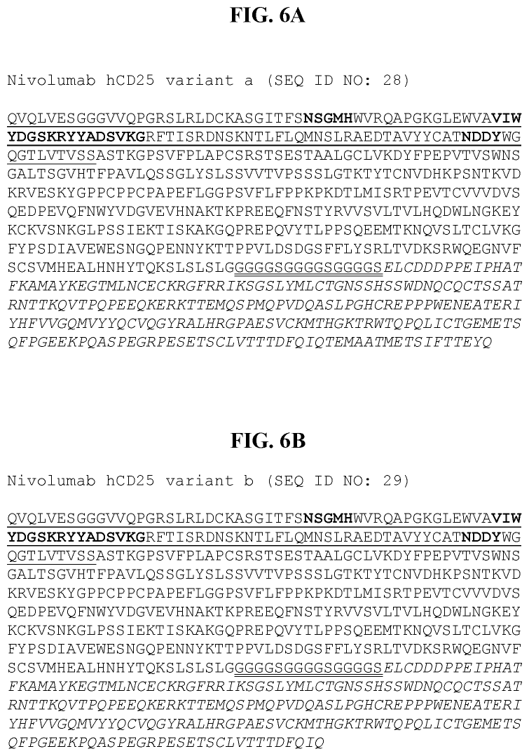

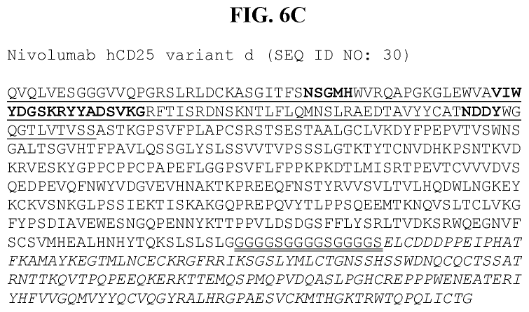

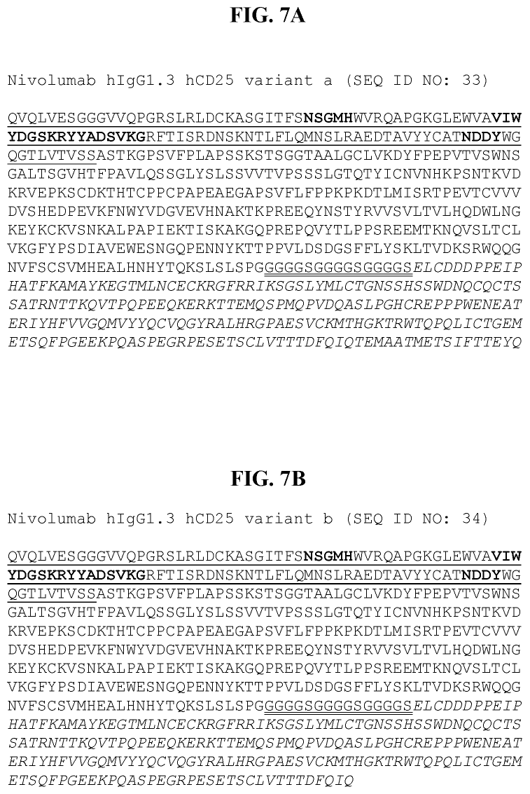

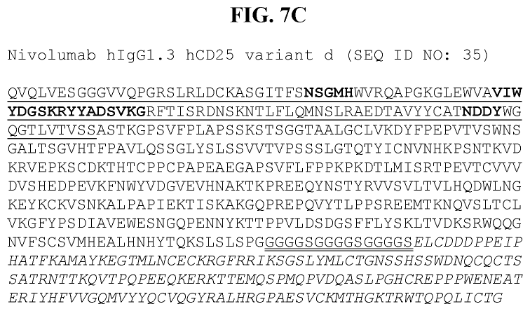

Exemplary mouse fusion proteins, comprising the heavy chain of anti-mPD1 mAb 4H2 fused to mCD25 variants a and b of the invention by a (G₄S)₃ linker, are provided at FIGS. 5A and 5B. Analogous human constructs comprising anti-hPD-1 mAb nivolumab heavy chain sequence fused to two variants of hCD25 by a (G₄S)₃ linker are provided at FIGS. 6A, 6B and 6C, and nivolumab variants comprising the effectorless hIgG1.3 constant domain are provided at FIGS. 7A, 7B and 7C.

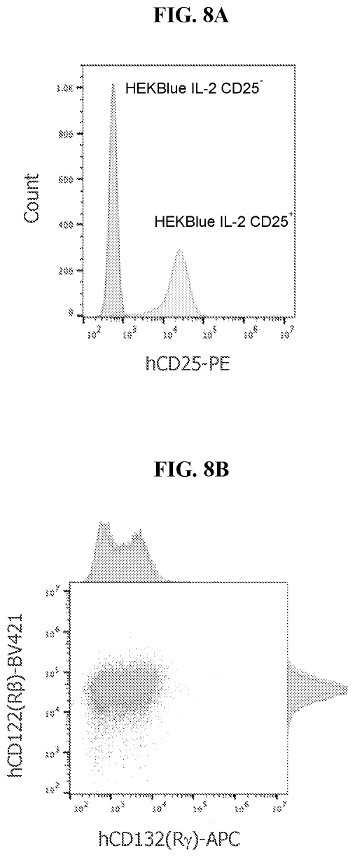

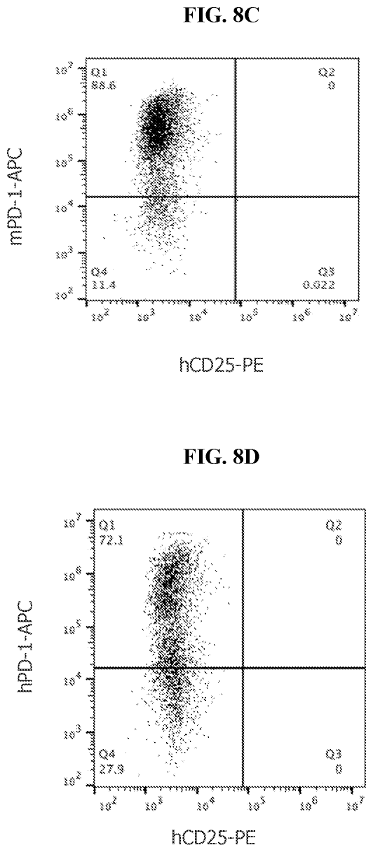

Cell lines were constructed to test the constructs of the present invention. The starting point was a commercial HEK-Blue IL-2 reporter cell line expressing alkaline phosphatase in response to IL-2 stimulation, enabling convenient colorimetric readout. The cell line was modified to delete the hCD25 gene, and then transduced to express either mPD-1 or hPD-1. See FIGS. 8A-8D. The resulting CD25⁻CD122⁺CD132⁺PD-1⁺ cells recapitulate the receptor expression pattern of the CD8+ Teff cells to be targeted in patients in that they express PD-1 but not CD25.

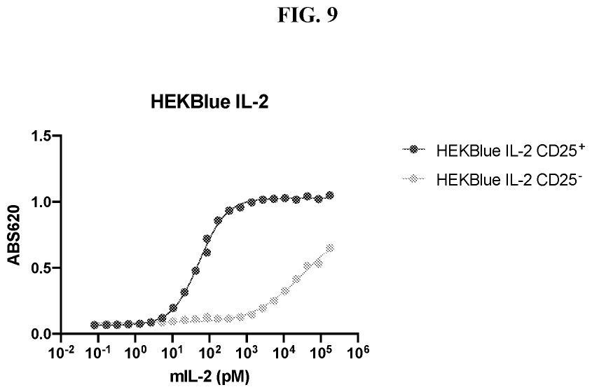

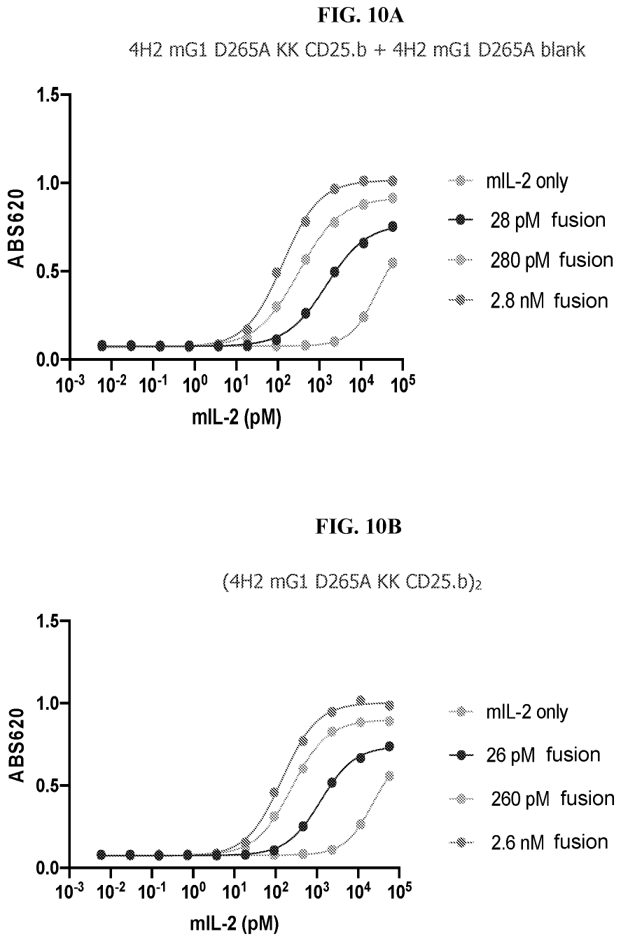

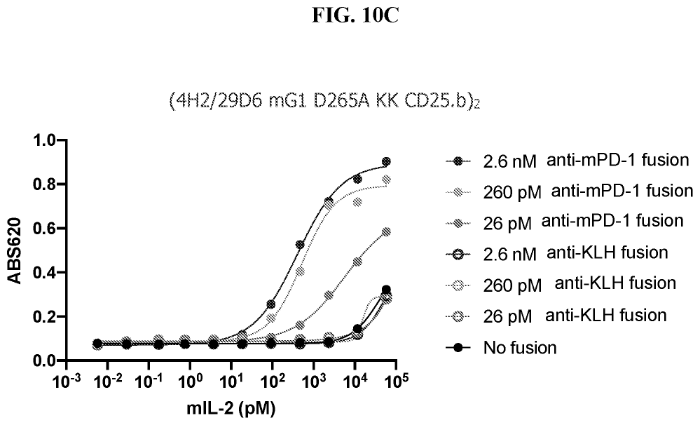

Comparison of the effects of IL-2 on CD25+ and CD25− cell lines demonstrated how important CD25 is to efficient IL-2 binding and signaling. See FIG. 9. FIGS. 10A and 10B, however, demonstrate that anti-PD-1 CD25 fusion constructs of the present invention, whether with CD25 on one antibody heavy chain or both, substantially restore IL-2 binding and signaling in a dose responsive manner. These effects were entirely dependent on PD-1 binding, as expected. See FIG. 10C.

These constructs were then tested on primary mouse splenocytes, which were sorted into CD8⁺CD25⁻ and CD4⁺CD25⁺ fractions. These fractions were exposed to varying levels of mIL-2 and phospho-STAT5 was measured. Results are provided at Table 3. As with the reporter cell line, the absence of CD25 reduces sensitivity to IL-2 by orders of magnitude.

TABLE 3
Percent Phospho-STAT5 Cells After IL-2 Stimulation

| [IL-2]: | 0 | 0.35 nM | 3.5 nM | 35 nM |
|---|---|---|---|---|
| CD8⁺ CD25⁻ | 0.9% | 1.5% | 12% | 63% |
| CD4⁺ CD25⁺ | 5.3% | 90% | 96% | 94% |

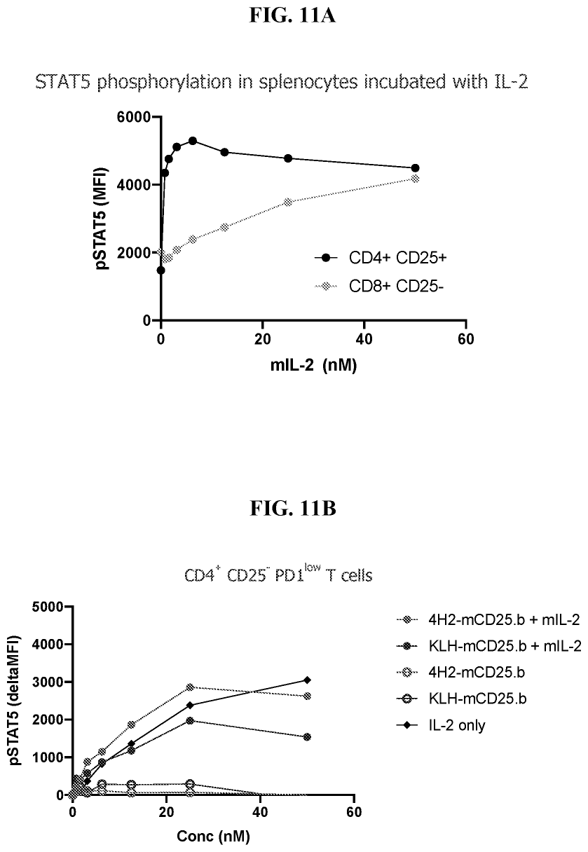

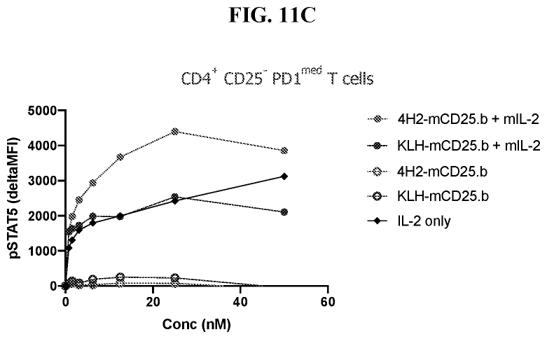

Similar results are provided graphically at FIG. 11A, where CD25⁻ cells are drastically less sensitive to IL-2. CD8⁺CD25⁻ and CD4⁺CD25⁻ mouse splenocytes were then sorted for PD-1 expression, to generate one pool of CD8⁺CD25⁻PD-1^{low} T cells and another of CD4⁺CD25⁻ PD-1^{med} T cells. Both pools were titrated with mIL-2 in the presence or absence of a mixture of mAb 4H2-mCD25 fusion construct and mIL-2. Results are provided at FIGS. 11B and 11C. The results show higher IL-2 mediated signaling in cells with higher PD-1 expression, confirming the ability of an anti-PD-1−CD25 fusion construct of the present invention to enhance IL-2 signaling preferentially in cells expressing PD-1 at higher levels.

Taken together these results in mouse models suggest that the anti-PD-1 CD25 fusion constructs of the present invention can be used to supplement the CD25 missing from PD-1⁺CD25⁻ cells, like T_{eff} in human TIL, and induce a more robust anti-tumor response driven by endogenous IL-2 without the need for systemic administration of a toxic IL-2 construct.

### Alternative Targeting Moieties and Disease Indications

The methods and constructs of the present invention are not limited to constructs comprising anti-PD-1 antibody binding domains. CD25 fusion construct comprising antibodies to other surface markers specific for anti-tumor CD8+ T, CD4+ T and NK cells may be used. Such surface markers will ideally be found on CD8⁺ effector T cells (T_{eff}) expressing CD122 (IL-2Rβ) and CD132 (IL-2Rγ), but not CD25, such that the CD25 fusion construct of the present invention can enhance IL-2 signaling. The ideal surface marker would not be found on T_{regs}. Exemplary alternative cell surface markers for use in the present invention include NKG2a, CD8a, FcRL6, CRTAM and LAG3.

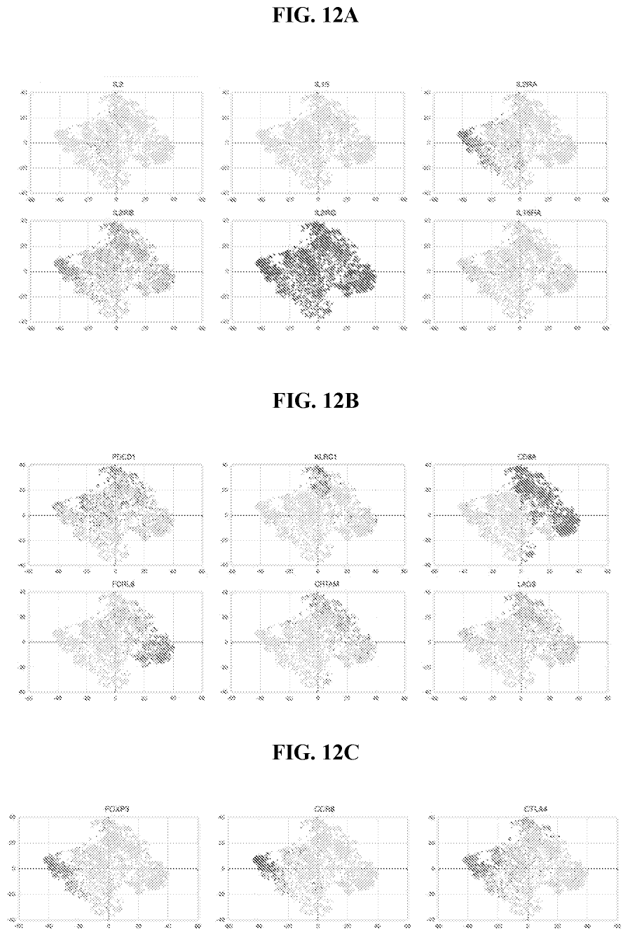

FIGS. 12A-12C show gene expression data in human NSCLC samples. FIG. 12A identifies populations of cells expressing IL2RB and IL2RG, the genes encoding the beta and gamma subunits (IL-2Rβ and IL-2Rγ) of the IL-2 receptor. Expression of these subunits is critical for treatment with the fusion constructs of the present invention, which deliver the missing IL-2Rα (CD25) subunit to complete the high affinity IL-2 receptor complex on target cells. Cells with low expression of IL2RA (encoding IL-2Rα) would be most likely to benefit from IL-2R supplementation by the methods and constructs of the present invention.

FIG. 12C shows populations of cells expressing FOXP3, CCR8 and CTLA4, which are markers for immunosuppressive regulatory T cells (T_{regs}). The methods of the present invention are intended to enhance IL-2 signaling in anti-tumor T_{eff} cells, to tip the balance between IL-2 signaling from T_{regs} to T_{eff}. Consequently, alternative targeting moieties of the present invention should not be expressed on T_{regs}.

Consequently, alternative targeting moieties of the present invention would ideally bind selectively to IL2RB⁺IL2RG⁺IL2RA⁻FOXP3⁻CCR8⁻CTLA4⁻ T cells. FIG. 12B shows the expression pattern for selected alternative targeting moieties of the present invention that meet these selection criteria in the tested NSCLC samples. Preferred targets include PD-1, NKG2a, CD8a, FcRL6, CRTAM and LAG3. As seen in FIGS. 12A, 12B and 12C, the genes encoding these surface markers are selectively expressed on NSCLC cells that express the beta and gamma subunits of IL-2 receptor, lack expression of the alpha subunit, and that are not T_{regs}.

Human PD-1 (programmed cell death protein 1) is encoded by the gene PDCD1 (NCBI Gene ID No: 5133), and is also known as PD1, PD-1, CD279, SLEB2, hPD-1, hPD-1, and hSLE1. Protein and nucleic acid sequences for the precursor protein are found, e.g., at GenBank Accession Nos: NP_005009.2 and NM_005018.3, respectively. In one embodiment, the constructs of the present invention comprise a targeting moiety that specifically binds to PD-1, such as an anti-PD-1 antibody. An exemplary anti-PD-1 antibody is OPDIVO®/nivolumab (BMS-936558) or an antibody that comprises the CDRs or variable regions of one of antibodies 17D8, 2D3, 4H1, 5C4, 7D3, 5F4 and 4A11 described in WO 2006/121168. In certain embodiments, an anti-PD-1 antibody is MK-3475 (KEYTRUDA®/pembrolizumab/formerly lambrolizumab) described in WO 2012/145493; AMP-514/MEDI-0680 described in WO 2012/145493; and CT-011 (pidilizumab; previously CT-AcTibody or BAT; see, e.g., Rosenblatt et al. (2011) J. Immunotherapy 34:409). Further known PD-1 antibodies and other PD-1 inhibitors include those described in WO 2009/014708, WO 03/099196, WO 2009/114335, WO 2011/066389, WO 2011/161699, WO 2012/145493, U.S. Pat. Nos. 7,635,757 and 8,217,149, and U.S. Patent Publication No. 2009/0317368. Any of the anti-PD-1 antibodies disclosed in WO 2013/173223 may also be used. Additional anti-PD-1 antibodies may be raised by conventional methods, including but not limited to humanized transgenic mice and phage display.

Human NKG2a is encoded by the gene KLRC1 (NCBI Gene ID No: 3821; killer cell lectin like receptor C1), and is also known as NKG2 and CD159A. Protein and nucleic acid sequences for the protein are found, e.g., at GenBank Accession Nos: NP_002250.2 and NM_002259.5, respectively. In one embodiment, the constructs of the present invention comprise a targeting moiety that specifically binds to NKG2a, such as an anti-NKG2a antibody. An exemplary anti-NKG2a antibody is BMS-986315. See WO 2020/102501. Another exemplary anti-NKG2a antibody is monalizumab (IPH2201), for which the heavy and light chain sequences are publicly available at pINN publication WHO Drug Information (2015) Vol. 29:2.

Human CD8a (CD8 alpha chain) is encoded by the gene CD8A (NCBI Gene ID No: 925), and is also known as CD8, p32 and Leu2. Protein and nucleic acid sequences for the precursor protein are found, e.g., at GenBank Accession Nos: NP_001759.3 and NM_001768.7, respectively. In one embodiment, the constructs of the present invention comprise a targeting moiety that specifically binds to CD8a, such as an anti-CD8a antibody. Exemplary anti-CD8a antibodies are provided as mAbs OKT8 and 51.1 (FIGS. 25-28) in U.S. Pat. No. 10,428,155; and also at FIG. 16 of WO 2020/060924. Additional anti-CD8 mAbs are provided at WO 2019/023148 and at U.S. Pat. No. 10,072,080.

Human FcRL6 (Fc receptor like 6) is encoded by the gene FCRL6 (NCBI Gene ID No: 343413), and is also known as FcRH6. Protein and nucleic acid sequences for the precursor protein are found, e.g., at GenBank Accession Nos: NP_001004310.2 and NM_001004310.3, respectively. In one embodiment, the constructs of the present invention comprise a targeting moiety that specifically binds to FcRL6, such as an anti-FcRL6 antibody. Exemplary anti-FcRL6 antibodies 1D8 and 7B7 are described at Shreeder et al. (2010) J. Immunol. 185:23 and Shreeder et al. (2008) Eur. J. Immunol. 38:3159. See also WO 2019/094743.

Human CRTAM (cytotoxic and regulatory T cell molecule) is encoded by the gene CRTAM (NCBI Gene ID No: 56253), and is also known as CD355. Protein and nucleic acid sequences for the precursor protein are found, e.g., at GenBank Accession Nos: NP_062550.2 and NM_019604.4, respectively. In one embodiment, the constructs of the present invention comprise a targeting moiety that specifically binds to CRTAM, such as an anti-CRTAM antibody. An exemplary anti-CRTAM is 5A11 at WO 2019/086878. See also WO 2009/029883.

Human LAG3 (lymphocyte activation gene 3) is encoded by the gene LAG3 (NCBI Gene ID No: 3902), and is also known as CD223. Protein and nucleic acid sequences for the precursor protein are found, e.g., at GenBank Accession Nos: NP_002277.4 and NM_002286.6, respectively. In one embodiment, the constructs of the present invention comprise a targeting moiety that specifically binds to LAG3, such as an anti-LAG3 antibody. Examples of anti-LAG3 antibodies include antibodies comprising the CDRs or variable regions of antibodies 25F7, 26H10, 25E3, 8B7, 11F2 or 17E5, which are described in U.S. Patent Publication No. US 2011/0150892 and WO 2014/008218. In one embodiment, an anti-LAG-3 antibody is relatlimab (BMS-986016). Other art recognized anti-LAG-3 antibodies that can be used include IMP731 described in US 2011/007023. IMP701, referred to as LAG525 in humanized form, as described and claimed in nucleic acid form in U.S. Pat. No. 10,711,060, may also be used. Agonist mAb IMP761 (mAb 13E2) may also be used. WO 2017/037203. Additional anti-LAG3 antibodies may be raised by conventional methods, including but not limited to humanized transgenic mice and phage display.

The same targets identified for use as targeting moieties in the methods and constructs of the present invention using NCSLC samples (Guo et al. (2018) Nat. Med. 24:978) are also preferentially expressed in the desired T cell populations in other cancers. Datasets were analyzed for the following cancers: breast cancer (Savas et al. (2018) Nat. Med. 24:986); melanoma (Li et al. (2019) Cell 176:775; Sadi-Feldman et al. (2018) Cell 175:998); metastatic melanoma (Tirosh et al. (2016) Science 352:189); colon cancer (Zhang et al. (2020) Cell 181:442); liver cancer (Zheng et al. (2017) Cell 169:1342); colorectal cancer (Zhang et al. (2018) Nature 564:268). As a consequence, the methods of the present invention using PD-1, NKG2a, CD8a, FcRL6, CRTAM and LAG3 as targeting moieties may find particular applicability in treating NSCLC, liver cancer, breast cancer, colorectal cancer (CRC), metastatic melanoma, colon cancer, and melanoma. In selected embodiments, methods and constructs of the present invention are used in treating NSCLC, liver cancer, breast cancer, such as specifically NSCLC.

### Tumor-Targeted Antigen Binding

In various embodiments, the anti-PD-1 CD25 fusion construct of the present invention is modified to selectively block antigen binding in tissues and environments where antigen binding would be detrimental, but allow antigen binding where it would be beneficial. In one embodiment, a blocking peptide “mask” is generated that specifically binds to the antigen binding surface of the anti-PD-1 antibody and interferes with antigen binding, which mask is linked to each of the binding arms of the antibody by a peptidase cleavable linker. See Int'l Pat. App. Pub. No. WO 17/011580 to CytomX. Such constructs are useful for treatment of cancers in which protease levels are greatly increased in the tumor microenvironment compared with non-tumor tissues. Selective cleavage of the cleavable linker in the tumor microenvironment allows disassociation of the masking/blocking peptide, enabling antigen binding selectively in the tumor, rather than in peripheral tissues in which antigen binding might cause unwanted side effects.

Alternatively, in a related embodiment, a bivalent binding compound (“masking ligand”) comprising two antigen binding domains is developed that binds to both antigen binding surfaces of the (bivalent) antibody and interfere with antigen binding, in which the two binding domains masks are linked to each other (but not the antibody) by a cleavable linker, for example cleavable by a peptidase. See, e.g., Int'l Pat. App. Pub. No. WO 2010/077643 to Tegopharm Corp. Masking ligands may comprise, or be derived from, the antigen to which the antibody is intended to bind, or may be independently generated. Such masking ligands are useful for treatment of cancers in which protease levels are greatly increased in the tumor microenvironment compared with non-tumor tissues. Selective cleavage of the cleavable linker in the tumor microenvironment allows disassociation of the two binding domains from each other, reducing the avidity for the antigen-binding surfaces of the antibody. The resulting dissociation of the masking ligand from the antibody enables antigen binding selectively in the tumor, rather than in peripheral tissues in which antigen binding might cause unwanted side effects.

In yet further embodiments, the anti-PD-1 CD25 fusion construct of the present invention comprises an antibody that preferentially binds to PD-1 at the pH of the tumor microenvironment (e.g. pH 6.0-6.5) rather than the pH of the periphery (e.g. pH 7.0-7.5). Int'l Pat. App. Pub. No. WO 20/214748; WO 20/092155.

### Nucleic Acid Molecules Encoding Anti-PD-1 CD25 Fusion Constructs of the Invention

Another aspect of the present disclosure pertains to isolated nucleic acid molecules that encode any of the anti-PD-1 CD25 fusion constructs of the present invention, including the heavy and/or light chains of the anti-PD-1 antibody portion of the fusion constructs. The nucleic acids may be present in whole cells, in a cell lysate, or in a partially purified or substantially pure form. The nucleic acid can be, for example, DNA or RNA, and may or may not contain intronic sequences. In certain embodiments, the DNA is genomic DNA, cDNA, or synthetic DNA, i.e., DNA synthesized in a laboratory, e.g., by the polymerase chain reaction or by chemical synthesis. In some embodiments the heavy and light chain sequences are encoded in the same nucleic acid, whereas in other constructs the heavy and light chains are encoded by separate nucleic acids.

### Reduced Fucosylation, Nonfucosylation and Hypofucosylation

The interaction of anti-PD-1 CD25 fusion constructs of the present invention with FcγRs can also be enhanced by modifying the glycan moiety attached to each Fc fragment at the N297 residue. In particular, the absence of core fucose residues strongly enhances ADCC via improved binding of IgG to activating FcγRIIIA without altering antigen binding or CDC. Natsume et al. (2009) Drug Des. Devel. Ther. 3:7. There is convincing evidence that afucosylated tumor-specific antibodies translate into enhanced therapeutic activity in mouse models in vivo. Nimmerjahn & Ravetch (2005) Science 310:1510; Mossner et al. (2010) Blood 115:4393.

Modification of antibody glycosylation can be accomplished by, for example, expressing the antibody in a host cell with altered glycosylation machinery. Antibodies with reduced or eliminated fucosylation, which exhibit enhanced ADCC, are particularly useful in the methods of the present invention. Cells with altered glycosylation machinery have been described in the art and can be used as host cells in which to express recombinant antibodies of this disclosure to thereby produce an antibody with altered glycosylation. For example, the cell lines Ms704, Ms705, and Ms709 lack the fucosyltransferase gene, FUT8 (α-(1,6) fucosyltransferase (see U.S. Pat. App. Publication No. 20040110704; Yamane-Ohnuki et al. (2004) Biotechnol. Bioeng. 87: 614), such that antibodies expressed in these cell lines lack fucose on their carbohydrates. As another example, EP 1176195 also describes a cell line with a functionally disrupted FUT8 gene as well as cell lines that have little or no activity for adding fucose to the N-acetylglucosamine that binds to the Fc region of the antibody, for example, the rat myeloma cell line YB2/0 (ATCC CRL 1662). PCT Publication WO 03/035835 describes a variant CHO cell line, Lec13, with reduced ability to attach fucose to Asn(297)-linked carbohydrates, also resulting in hypofucosylation of antibodies expressed in that host cell. See also Shields et al. (2002) J. Biol. Chem. 277:26733. Antibodies with a modified glycosylation profile can also be produced in chicken eggs, as described in PCT Publication No. WO 2006/089231. Alternatively, antibodies with a modified glycosylation profile can be produced in plant cells, such as Lemna. See e.g. U.S. Publication No. 2012/0276086. PCT Publication No. WO 99/54342 describes cell lines engineered to express glycoprotein-modifying glycosyl transferases (e.g., beta(1,4)-N-acetylglucosaminyltransferase III (GnTIII)) such that antibodies expressed in the engineered cell lines exhibit increased bisecting GlcNAc structures which results in increased ADCC activity of the antibodies. See also Umaña et al. (1999) Nat. Biotech. 17:176. Alternatively, the fucose residues of the antibody may be cleaved off using a fucosidase enzyme. For example, the enzyme alpha-L-fucosidase removes fucosyl residues from antibodies. Tarentino et al. (1975) Biochem. 14:5516. Antibodies with reduced fucosylation may also be produced in cells harboring a recombinant gene encoding an enzyme that uses GDP-6-deoxy-D-lyxo-4-hexylose as a substrate, such as GDP-6-deoxy-D-lyxo-4-hexylose reductase (RMD), as described at U.S. Pat. No. 8,642,292. Alternatively, cells may be grown in medium containing fucose analogs that block the addition of fucose residues to the N-linked glycan or a glycoprotein, such as antibody, produced by cells grown in the medium. U.S. Pat. No. 8,163,551; WO 09/135181.

Because afucosylated antibodies exhibit greatly enhanced ADCC compared with fucosylated antibodies, antibody preparations need not be completely free of fucosylated heavy chains to be useful in the methods of the present invention. Residual levels of fucosylated heavy chains will not significantly interfere with the ADCC activity of a preparation substantially of afucosylated heavy chains. Antibodies produced in conventional CHO cells, which are fully competent to add core fucose to N-glycans, may nevertheless comprise from a few percent up to 15% afucosylated antibodies. Afucosylated antibodies may exhibit ten-fold higher affinity for CD16, and up to 30- to 100-fold enhancement of ADCC activity, so even a small increase in the proportion of afucosylated antibodies may drastically increase the ADCC activity of a preparation. Any preparation comprising more afucosylated antibodies than would be produced in normal CHO cells in culture may exhibit some level of enhanced ADCC. Such antibody preparations are referred to herein as preparations having reduced fucosylation. Depending on the original level of afucosylation obtained from normal CHO cells, reduced fucosylation preparations may comprise as little as 50%, 30%, 20%, 10% and even 5% afucosylated antibodies. Reduced fucosylation is functionally defined as preparations exhibiting two-fold or greater enhancement of ADCC compared with antibodies prepared in normal CHO cells, and not with reference to any fixed percentage of afucosylated species.

In other embodiments the level of nonfucosylation is structurally defined. As used herein, nonfucosylated antibody preparations are antibody preparations comprising greater than 95% afucosylated antibody heavy chains, including 100%. Hypofucosylated antibody preparations are antibody preparations comprising less than or equal to 95% heavy chains lacking fucose, e.g. antibody preparations in which between 80 and 95% of heavy chains lack fucose, such as between 85 and 95%, and between 90 and 95%. Unless otherwise indicated, hypofucosylated refers to antibody preparations in which 80 to 95% of heavy chains lack fucose, nonfucosylated refers to antibody preparations in which over 95% of heavy chains lack fucose, and “hypofucosylated or nonfucosylated” refers to antibody preparations in which 80% or more of heavy chains lack fucose.

In some embodiments, hypofucosylated or nonfucosylated antibodies are produced in cells lacking an enzyme essential to fucosylation, such as alpha1,6-fucosyltransferase encoded by FUT8 (e.g. U.S. Pat. No. 7,214,775), or in cells in which an exogenous enzyme partially depletes the pool of metabolic precursors for fucosylation (e.g. U.S. Pat. No. 8,642,292), or in cells cultured in the presence of a small molecule inhibitor of an enzyme involved in fucosylation (e.g. WO 09/135181).

The level of fucosylation in an antibody preparation may be determined by any method known in the art, including but not limited to gel electrophoresis, liquid chromatography, and mass spectrometry. Unless otherwise indicated, for the purposes of the present invention, the level of fucosylation in an antibody preparation is determined by hydrophilic interaction chromatography (or hydrophilic interaction liquid chromatography, HILIC). To determine the level of fucosylation of an antibody preparation, samples are denatured treated with PNGase F to cleave N-linked glycans, which are then analyzed for fucose content. LC/MS of full-length antibody chains is an alternative method to detect the level of fucosylation of an antibody preparation, but mass spectroscopy is inherently less quantitative.

### Pharmaceutical Compositions

Anti-PD-1 CD25 fusion constructs of the present invention may be constituted in a composition, e.g., a pharmaceutical composition, containing the binding protein, for example an antibody or a fragment thereof, and a pharmaceutically acceptable carrier. As used herein, a “pharmaceutically acceptable carrier” includes any and all solvents, dispersion media, coatings, antibacterial and antifungal agents, isotonic and absorption delaying agents, and the like that are physiologically compatible. Preferably, the carrier is suitable for intravenous, subcutaneous, intramuscular, parenteral, spinal or epidermal administration (e.g., by injection or infusion). A pharmaceutical composition of the invention may include one or more pharmaceutically acceptable salts, anti-oxidant, aqueous and non-aqueous carriers, and/or adjuvants such as preservatives, wetting agents, emulsifying agents and dispersing agents.

Actual dosage levels of the active ingredients in the pharmaceutical compositions of the present invention may be varied so as to obtain an amount of the active ingredient that is effective to achieve the desired therapeutic response for a particular patient, composition, and mode of administration, without being unduly toxic to the patient. The selected dosage level will depend upon a variety of pharmacokinetic factors including the activity of the particular compositions of the present invention employed, the route of administration, the time of administration, the rate of excretion of the particular compound being employed, the duration of the treatment, other drugs, compounds and/or materials used in combination with the particular compositions employed, the age, sex, weight, condition, general health and prior medical history of the patient being treated, and like factors well known in the medical arts. One of ordinary skill in the art would be able to determine appropriate dosages based on such factors as the subject's size, the severity of the subject's symptoms, and the particular composition or route of administration selected. A composition of the present invention can be administered via one or more routes of administration using one or more of a variety of methods well known in the art.

### Therapeutic Uses and Methods of the Invention

This disclosure provides methods for cancer immunotherapy, e.g. potentiating an endogenous immune response in a subject afflicted with a cancer so as to thereby treat the subject, which method comprises administering to the subject a therapeutically effective amount of any of the anti-PD-1 CD25 fusion constructs described herein. In preferred embodiments of the present immunotherapeutic methods, the subject is a human.

Examples of other cancers that may be treated using the immunotherapeutic methods of the disclosure include bone cancer, pancreatic cancer, skin cancer, cancer of the head or neck, breast cancer, lung cancer, cutaneous or intraocular malignant melanoma, renal cancer, uterine cancer, ovarian cancer, colorectal cancer, colon cancer, rectal cancer, cancer of the anal region, stomach cancer, testicular cancer, uterine cancer, carcinoma of the fallopian tubes, carcinoma of the endometrium, carcinoma of the cervix, carcinoma of the vagina, carcinoma of the vulva, cancer of the esophagus, cancer of the small intestine, cancer of the endocrine system, cancer of the thyroid gland, cancer of the parathyroid gland, cancer of the adrenal gland, sarcoma of soft tissue, cancer of the urethra, cancer of the penis, a hematological malignancy, solid tumors of childhood, lymphocytic lymphoma, cancer of the bladder, cancer of the kidney or ureter, carcinoma of the renal pelvis, neoplasm of the central nervous system (CNS), primary CNS lymphoma, tumor angiogenesis, spinal axis tumor, brain stem glioma, pituitary adenoma, Kaposi's sarcoma, epidermoid cancer, squamous cell cancer, environmentally induced cancers including those induced by asbestos, metastatic cancers, and any combinations of said cancers. In preferred embodiments, the cancer is selected from MEL, RCC, squamous NSCLC, non-squamous NSCLC, CRC, CRPC, squamous cell carcinoma of the head and neck, and carcinomas of the esophagus, ovary, gastrointestinal tract and breast. The present methods are also applicable to treatment of metastatic cancers.

Other cancers include hematologic malignancies including, for example, multiple myeloma, B-cell lymphoma, Hodgkin lymphoma/primary mediastinal B-cell lymphoma, non-Hodgkin's lymphomas, acute myeloid lymphoma, chronic myelogenous leukemia, chronic lymphoid leukemia, follicular lymphoma, diffuse large B-cell lymphoma, Burkitt's lymphoma, immunoblastic large cell lymphoma, precursor B-lymphoblastic lymphoma, mantle cell lymphoma, acute lymphoblastic leukemia, mycosis fungoides, anaplastic large cell lymphoma, T-cell lymphoma, and precursor T-lymphoblastic lymphoma, and any combinations of said cancers.

### Combination Therapy

In certain embodiments of these methods for treating a cancer patient, the anti-PD-1 CD25 fusion construct of the present invention is administered to the subject as monotherapy, whereas in other embodiments, stimulation or blockade of immunomodulatory targets may be effectively combined with standard cancer treatments, including chemotherapeutic regimes, radiation, surgery, hormone deprivation and angiogenesis inhibitors.

Anti-PD-1 CD25 fusion constructs of the present invention may also be used in combination with other immunomodulatory agents, such as antibodies against other immunomodulatory receptors or their ligands. Several other co-stimulatory and inhibitory receptors and ligands that regulate T cell responses have been identified. Examples of stimulatory receptors include Inducible T cell Co-Stimulator (ICOS), CD137 (4-1BB), CD134 (OX40), CD27, Glucocorticoid-Induced TNFR-Related protein (GITR), and HerpesVirus Entry Mediator (HVEM), whereas examples of inhibitory receptors include Programmed Death-1 (PD-1), B and T Lymphocyte Attenuator (BTLA), T cell Immunoglobulin and Mucin domain-3 (TIM-3), Lymphocyte Activation Gene-3 (LAG-3), adenosine A2a receptor (A2aR), Killer cell Lectin-like Receptor G1 (KLRG-1), Natural Killer Cell Receptor 2B4 (CD244), CD160, T cell Immunoreceptor with Ig and ITIM domains (TIGIT), and the receptor for V-domain Ig Suppressor of T cell Activation (VISTA). Mellman et al. (2011) Nature 480:480; Pardoll (2012) Nat. Rev. Cancer 12: 252; Baitsch et al. (2012) PloS One 7:e30852. These receptors and their ligands provide targets for therapeutics designed to stimulate, or prevent the suppression, of an immune response so as to thereby attack tumor cells. Weber (2010) Semin. Oncol. 37:430; Flies et al. (2011) Yale J. Biol. Med. 84:409; Mellman et al. (2011) Nature 480:480; Pardoll (2012) Nat. Rev. Cancer 12:252. Stimulatory receptors or receptor ligands are targeted by agonist agents, whereas inhibitory receptors or receptor ligands are targeted by blocking agents. Among the most promising approaches to enhancing immunotherapeutic anti-tumor activity is the blockade of so-called “immune checkpoints,” which refer to the plethora of inhibitory signaling pathways that regulate the immune system and are crucial for maintaining self-tolerance and modulating the duration and amplitude of physiological immune responses in peripheral tissues in order to minimize collateral tissue damage. See e.g. Weber (2010) Semin. Oncol. 37:430; Pardoll (2012) Nat. Rev. Cancer 12:252. Because many of the immune checkpoints are initiated by ligand-receptor interactions, they can be readily blocked by antibodies or modulated by recombinant forms of ligands or receptors.

The present invention is further illustrated by the following examples, which should not be construed as limiting. The contents of all figures and all references, patents and published patent applications cited throughout this application are expressly incorporated herein by reference.

### EXAMPLE 1

### Binding of Truncated Anti-PD-1−mCD25 Variant Fusion Proteins to mIL-2

Surface plasmon resonance spectroscopy (SPR) was used to measure binding of selected truncated mCD25 variants to mIL-2 when present in a fusion construct with anti-mPD-1 mAb 4H2. Truncations of mCD25 are presented at FIG. 3A.

Unless otherwise indicated, binding kinetics were determined with a BIACORE® SPR surface plasmon resonance spectrometer (Biacore AB, Uppsala, Sweden). The mouse IL-2 binding affinity was determined for mPD1−mCD25 variants of the present invention using a Biacore™ T200 instrument. The assay temperature was 37° C. and the running buffer was HEPES buffered saline pH 7.4 supplemented with 0.05% (v/v) Tween-20 and 1 g/L BSA. Purified mPD1−mCD25 variants were captured on a Biacore™ CM4 chip with immobilized anti-mouse IgG polyclonal capture antibody. Mouse IL-2 was injected as analyte in a six-membered, three-fold dilution series with 250 nM top concentration and a duplicate injection at 83 nM. Between cycles, the capture surface was regenerated for three minutes with 10 mM Glycine pH 1.7. Double-referenced sensorgrams were fitted to a 1:1 Langmuir binding model with mass transport to determine equilibrium dissociation constants (K_{D}), as well as association (kₐ) and dissociation (k_{d}) rate constants where appropriate. Both the full-length construct and CD25.b bind mIL-2 with a K_{D} of 14 nM.

Binding analyses were also performed with an Octet HTX. Briefly, mPD1−mCD25 variants of the present invention were produced and captured on anti-mouse Fc tips. Mouse IL-2 incubated as analyte at 0.6 μM concentration at 25° C. HEPES buffered saline pH 7.4 containing 150 mM NaCl, 0.05% Tween and 0.5% BSA was used for these experiments. Data are provided as sensorgrams at FIG. 4. Full length mCD25 ECD, variant a, binds to mIL-2, as does variant b, but variant c, comprising only the sushi 1 domain, does not.

Additional modified hCD25 variants d, e and f were also prepared, with sequences as provided at FIG. 3B and at SEQ ID NOs: 14, 15 and 16, respectively. Octet binding experiments demonstrated that like variant c, variants e and f bound poorly to hCD25. SPR experiments were performed to determine the binding parameters for variant a, variant b and variant d, with results provided at Table 4. All variants tested bound with K_{D} of 12 to 14 nM. Taken as a whole these results, consistent with the mouse data provided at FIG. 4, demonstrate that all sushi 2 domain residues and all structurally defined sushi 1 domain residues are necessary, and sufficient, for a construct that binds to hCD25, with human variant d as the minimal essential construct among those tested.

TABLE 4
Summary of the Sequence Listing

| Antibody | Antigen | ka (1/Ms) | kd (1/s) | K_{D} (M) |
|---|---|---|---|---|
| GS_hCD25.a | hIL2- | 5.2E+06 | 7.5E−02 | 1.4E−08 |
| | Miltenyi | | | |
| GS_hCD25.b | hIL2- | 1.1E+07 | Fast | 1.2E−08 |
| | Miltenyi | | | |
| GS_hCD25.d | hIL2- | 5.4E+06 | 6.3E−02 | 1.2E−08 |
| | Miltenyi | | | |

### EXAMPLE 2

### Generation of hCD25⁻hCD122⁺hCD132⁺mPD-1⁺ and hCD25⁻hCD122⁺hCD132⁺hPD-1⁺ HEK-Blue™ IL-2 Reporter Cell Lines

Reporter cell lines were constructed to test the anti-PD-1−CD25 constructs of the present invention. HEK-Blue™ IL-2 cells were modified to delete hCD25, and to add either mPD-1 or hPD-1, as follows. Briefly, cell lines were derived from HEK-Blue™ IL-2 reporter cells engineered to generate and chromogenic alkaline phosphatase signal reflecting hIL-2 signaling. InvivoGen, San Diego, Calif., USA. The cells are engineered to express hCD25 (IL-2Rα), hCD122 (IL-2Rβ) and hCD132 (IL-2Rγ), which are the three subunits of the IL-2 receptor, as well as hJAK3, hSTAT5, and a STAT5-inducible SEAP (secreted embryonic alkaline phosphatase) reporter gene. Human CD25 was deleted from the HEK-Blue™ IL-2 reporter cells as follows. A plasmid encoding for guide RNAs targeting human CD25 gene, Cas9 enzyme and GFP was transfected into HEK-Blue™ IL-2 cells. After 24 hours, cells were sorted based on GFP expression, and GFP positive cells were cultured. CD25-positive and CD25-negative cells were sorted using a Sony MA900 cell sorter.

Deletion of the hCD25 gene was confirmed by FACS. See FIG. 8A. This hCD25⁻hCD122⁺hCD132⁺ reporter cell line was used in Example 3 (infra). The CD25 deleted cells were then transduced with vectors driving expression of mPD-1 or hPD-1, as follows. DNA sequences of human or mouse PD1 were cloned downstream to a promoter in a lentiviral vector. Lentiviral particles were produced using standard protocol. CD25-positive and CD25-negative HEK Blue IL-2 cells were transduced with human or mouse PD1 constructs. PD-1 expression was confirmed by FACS. See FIGS. 8C and 8D. The resulting CD25−PD-1+ reporter cell lines find use in evaluating the anti-PD-1−CD25 fusion constructs of the present invention.

### EXAMPLE 3

### IL-2 Stimulation of Reporter Cell Lines

The HEK-Blue™ IL-2 reporter cell line and the hCD25⁻ HEK-Blue™ IL-2 reporter cell line generated in Example 2 were titrated with mouse IL-2. Results are provided at FIG. 9.

The hCD25⁻ HEK-Blue™ IL-2 reporter cell line was then titrated with mIL-2 in the presence or absence of varying amounts of hemi-CD25 modified or fully CD25 modified mAb 4H2 fusion constructs. Results are provided at FIGS. 10A and 10B, respectively. Both constructs partially restored IL-2 signaling to CD25+ levels in a dose-dependent fashion. The mIL-2 titration with the fully CD25 modified 4H2 construct was repeated with an analogous fully CD25 modified anti-KLH antibody (29D6) construct. Results are provided at FIG. 10C.

### EXAMPLE 4

### Selective Stimulation of PD-1+ Primary T Cells

Mouse splenocytes were sorted into CD4+CD25+ and CD8+CD25− pools. The CD4+CD25+ and CD8+CD25− were titrated with mIL-2, and STAT5 phosphorylation was measured by flow cytometry. Results are provided at FIG. 11A. As with the HEK-Blue™ IL-2 reporter cell lines, lack of CD25 dramatically reduces IL-2 response.

In other experiments, CD4+ and CD8+ mouse splenocyte pools were stained at the same time for PD-1 and CD25 expression. CD25-negative cells were separated into two PD-1 expressing fractions (PD1^{low} and PD1^{medium}). Cells were incubated with a titration of mIL-2 in the presence and absence of fully CD25 modified 4H2 or fully CD25 modified anti-KLH mAb constructs, both alone and as mixtures with mIL-2. CD25 constructs with mouse IL-2 were pre-mixed at equal molar ratio for 30 minutes, and then incubated with the mouse cells for 40 minutes. Cells were then fixed, permeabilized and stained with anti-CD4, anti-CD8, anti-CD25, anti-PD1 and anti-phospho-STAT5 antibodies. Results are provided at FIGS. 11B and 11C.

### EXAMPLE 5

### Targets

Cell surface markers for use in targeting moieties in the methods and fusion constructs of the present invention were selected tumor samples for genes selectively expressed on T_{eff}, rather than T_{regs}, and specifically on T_{eff}, that also express the beta and gamma subunits of IL-2 receptor but not the alpha subunits. The constructs of the present invention deliver the missing alpha subunit to these T_{eff}, completing the trimeric (high affinity) IL-2 receptor complex, but will not bind to T_{regs}.

Single cell RNA sequencing data from tumor infiltrated lymphocytes (TIL) from NSCLC patients (Guo et al. (2018) Nat. Med. 24:978) were queried for expression of candidate target genes, T_{reg} markers FOXP3, CCR8 and CTLA-4, as well as IL2RA, IL2RB, IL2RG. Results, provided at FIGS. 12A-12C, demonstrate that PDCD1, KLRC1, CD8A, FCRL8, CRTAM and LAG3 are not expressed on T_{regs}, and are expressed on T_{eff} that also express IL2RB and IL2RG but not IL2RA.

Analogous analyses (not shown) were performed on single cell gene expression data from T cells from other tumor types, specifically liver cancer, breast cancer, colorectal cancer (CRC), metastatic melanoma, colon cancer, and melanoma. Savas et al. (2018) Nat. Med. 24:986); Li et al. (2019) Cell 176:775; Sadi-Feldman et al. (2018) Cell 175:998; Tirosh et al. (2016) Science 352:189; Zhang et al. (2020) Cell 181:442; Zheng et al. (2017) Cell 169:1342; Zhang et al. (2018) Nature 564:268. Results confirmed that the same targets found in NSCLC samples (PDCD1, KLRC1, CD8A, FCRL8, CRTAM and LAG3) would find use in treating all these cancers in addition to NSCLC.

TABLE 5
Summary of the Sequence Listing

| SEQ ID NO. | Description |
|---|---|
| 1 | mCD25 |
| 2 | mCD25 variant a |
| 3 | mCD25 variant b |
| 4 | mCD25 variant c |
| 5 | 4H2 heavy chain |
| 6 | 4H2 light chain |
| 7 | (G₄S)₃ linker |
| 8 | 4H2-mCD25 variant a |
| 9 | 4H2-mCD25 variant b |
| 10 | hCD25 |
| 11 | hCD25 variant a |
| 12 | hCD25 variant b |
| 13 | hCD25 variant c |
| 14 | hCD25 variant d |
| 15 | hCD25 variant e |
| 16 | hCD25 variant f |
| 17 | nivolumab CDRH1 |
| 18 | nivolumab CDRH2 |
| 19 | nivolumab CDRH3 |
| 20 | nivolumab CDRL1 |
| 21 | nivolumab CDRL2 |
| 22 | nivolumab CDRL3 |
| 23 | nivolumab heavy chain variable region |
| 24 | nivolumab light chain variable region |
| 25 | nivolumab heavy chain w/o C-terminal K |
| 26 | nivolumab heavy chain |
| 27 | nivolumab light chain |
| 28 | nivolumab-hCD25 variant a |
| 29 | nivolumab-hCD25 variant b |
| 30 | nivolumab-hCD25 variant d |
| 31 | nivolumab heavy chain IgG1.3 w/o C-terminal K |
| 32 | nivolumab heavy chain IgG1.3 |
| 33 | nivolumab IgG1.3 hCD25 variant a |
| 34 | nivolumab IgG1.3 hCD25 variant b |
| 35 | nivolumab IgG1.3 hCD25 variant d |
| 36 | pembrolizumab CDRH1 |
| 37 | pembrolizumab CDRH2 |
| 38 | pembrolizumab CDRH3 |
| 39 | pembrolizumab CDRL1 |
| 40 | pembrolizumab CDRL2 |
| 41 | pembrolizumab CDRL3 |
| 42 | pembrolizumab heavy chain variable region |
| 43 | pembrolizumab light chain variable region |
| 44 | pembrolizumab heavy chain w/o C-terminal K |
| 45 | pembrolizumab heavy chain |
| 46 | pembrolizumab light chain |
| 47 | pembrolizumab-hCD25 variant a |
| 48 | pembrolizumab-hCD25 variant b |
| 49 | pembrolizumab-hCD25 variant d |
| 50 | pembrolizumab heavy chain IgG1.3 w/o C-terminal K |
| 51 | pembrolizumab heavy chain IgG1.3 |
| 52 | pembrolizumab IgG1.3 hCD25 variant a |
| 53 | pembrolizumab IgG1.3 hCD25 variant b |
| 54 | pembrolizumab IgG1.3 hCD25 variant d |

With regard to antibody sequences, the Sequence Listing provides the sequences of the mature variable regions of the heavy and light chains, i.e. the sequences do not include signal peptides. Any signal sequence suitable for use in the production cell line being used may be used in production of the antibodies of the present invention. Heavy chain amino acid sequences may be provided without a C-terminal lysine residue, but in some embodiments such residue is encoded in the nucleic acid construct for the antibody.

### EQUIVALENTS

Those skilled in the art will recognize, or be able to ascertain using no more than routine experimentation, many equivalents of the specific embodiments disclosed herein. Such equivalents are intended to be encompassed by the following claims.

1. A polypeptide construct comprising a targeting moiety and a CD25 moiety, each of which comprises one or more amino acid sequences.

2. The polypeptide construct of claim 1 wherein the targeting moiety comprises an antibody, or an antigen binding fragment thereof.

3. The polypeptide construct of claim 2 wherein the targeting moiety comprises an antibody that binds specifically to a target selected from the group consisting of PD-1, NKG2a, CD8a, FcRL6, CRTAM and LAG3, or an antigen binding fragment thereof.

4. The polypeptide construct of claim 3 wherein the targeting moiety is an anti-PD-1 antibody or an antigen binding fragment thereof, wherein the anti-PD-1 antibody or antigen binding fragment comprises one or more heavy chains.

5. The polypeptide construct of claim 2, wherein the amino acid sequence of the CD25 moiety is appended to the C-terminus of at least one heavy chain of the antibody or antigen binding fragment thereof.

6. The polypeptide construct of claim 5 wherein the amino acid sequence of the CD25 moiety is appended to the C-terminus of both heavy chains of the anti-PD-1 antibody.

7. The polypeptide construct of claim 4, wherein the anti-PD-1 antibody or antigen binding fragment comprises nivolumab, pembrolizumab, a PD-1 binding fragment of nivolumab, or a PD-1 binding fragment of pembrolizumab.

8. The polypeptide construct of claim 7 wherein the PD-1 antibody comprises:
a. a heavy chain comprising a heavy chain variable domain comprising:
i. a CDRH1 of SEQ ID NO: 17;
ii. a CDRH2 of SEQ ID NO: 18;
iii. a CDRH3 of SEQ ID NO: 19; and
b. a light chain comprising a light chain variable domain comprising:
i. a CDRL1 of SEQ ID NO: 20;
ii. a CDRL2 of SEQ ID NO: 21;
iii. a CDRL3 of SEQ ID NO: 22.

9. The polypeptide construct of claim 8 comprising;
a. a heavy chain variable domain comprising the sequence of SEQ ID NO: 23; and
b. a light chain variable domain comprising the sequence of SEQ ID NO: 24.

10. The polypeptide construct of claim 9 comprising;
a. a heavy chain comprising the sequence of SEQ ID NO: 25; and
b. a light chain comprising the sequence of SEQ ID NO: 27.

11. The polypeptide construct of claim 1, wherein the CD25 moiety comprises the sequence of SEQ ID NO: 14.

12. The polypeptide construct of claim 11 wherein the human CD25 comprises the sequence of SEQ ID NO: 12.

13. The polypeptide construct of claim 12 wherein the human CD25 comprises the sequence of SEQ ID NO: 11.

14. The polypeptide construct of claim 1, further comprising a linker between the targeting moiety and CD25 moiety comprising the sequence of SEQ ID NO: 7.

15. The polypeptide construct of claim 14 comprising a first construct comprising:
a. a heavy chain comprising the sequence of SEQ ID NO: 28, 29 or 30; and
b. two light chains comprising the sequence of SEQ ID NO: 27;
or a second construct comprising:
a. two heavy chains comprising the same sequence, said sequence being selected from the group consisting of SEQ ID NO: 28, 29 or 30; and
b. two light chains each comprising the sequence of SEQ ID NO: 27;
or a third construct comprising:
a. a heavy chain comprising the sequence of SEQ ID NO: 25; and
b. a heavy chain comprising the sequence of SEQ ID NO: 28, 29 or 30; and
c. two light chains comprising the sequence of SEQ ID NO: 27.

16. (canceled)

17. (canceled)

18. The polypeptide construct of claim 15 wherein the sequence of both antibody heavy chains are modified by the knob-into-hole approach to promote heterodimeric heavy chain pairing.

19. A pharmaceutical composition comprising a polypeptide construct of claim 1.

20. A nucleic acid encoding one or more polypeptide chains of the polypeptide construct of claim 1.

21. An expression vector comprising the nucleic acid of claim 20.

22. A host cell comprising the expression vector of claim 21.

23. A method of making the polypeptide construct of claim 1 comprising:
a. culturing the host cell of claim 22 under conditions that allow production of the polypeptide construct; and
b. isolating the polypeptide construct.

24. A method of treating a disease in a human subject comprising administering to the subject the polypeptide construct of claim 1.

25. The method of claim 24 wherein the disease is cancer.

26. (canceled)

27. (canceled)

28. (canceled)

29. The method of claim 24 wherein human IL-2 is also administered to the subject.

30. A method of treating a disease in a human subject comprising:
a. obtaining tumor infiltrating lymphocytes (TIL) from the subject;
b. measuring IL-2 expression level in the TIL; and
c. administering the polypeptide construct of claim 1 only to subjects whose TIL exhibit IL-2 expression above a threshold level.

31. The method of claim 30 wherein the disease is cancer.

32. (canceled)


SEQUENCE LISTING (54 sequences)

SEQ ID NO: 1 - protein, 268 aa, Mus musculus
MEPRLLMLGFLSLTIVPSCRAELCLYDPPEVPNATFKALSYKNGTILNCECKRGFRRLKELVYMRCLGNSWSSNCQCTSNSHDKSRKQVTAQLEHQKEQQTTTDMQKPTQSMHQENLTGHCREPPPWKHEDSKRIYHFVEGQSVHYECIPGYKALQRGPAISICKMKCGKTGWTQPQLTCVDEREHHRFLASEESQGSRNSSPESETSCPITTTDFPQPTETTAMTETFVLTMEYKVAVASCLFLLISILLLSGLTWQHRWRKSRRTI

SEQ ID NO: 2 - protein, 215 aa, Mus musculus
ELCLYDPPEVPNATFKALSYKNGTILNCECKRGFRRLKELVYMRCLGNSWSSNCQCTSNSHDKSRKQVTAQLEHQKEQQTTTDMQKPTQSMHQENLTGHCREPPPWKHEDSKRIYHFVEGQSVHYECIPGYKALQRGPAISICKMKCGKTGWTQPQLTCVDEREHHRFLASEESQGSRNSSPESETSCPITTTDFPQPTETTAMTETFVLTMEYK

SEQ ID NO: 3 - protein, 198 aa, Mus musculus
ELCLYDPPEVPNATFKALSYKNGTILNCECKRGFRRLKELVYMRCLGNSWSSNCQCTSNSHDKSRKQVTAQLEHQKEQQTTTDMQKPTQSMHQENLTGHCREPPPWKHEDSKRIYHFVEGQSVHYECIPGYKALQRGPAISICKMKCGKTGWTQPQLTCVDEREHHRFLASEESQGSRNSSPESETSCPITTTDFPQP

SEQ ID NO: 4 - protein, 103 aa, Mus musculus
SYKNGTILNCECKRGFRRLKELVYMRCLGNSWSSNCQCTSNSHDKSRKQVTAQLEHQKEQQTTTDMQKPTQSMHQENLTGHCREPPPWKHEDSKRIYHFVEGQ

SEQ ID NO: 5 - protein, 443 aa, Mus musculus
QVQLKESGPGLVQPSQTLSLTCTVSGFSLTSYNVHWVRQPPGKGLEWMGGMRYNEDTSYNSALKSRLSISRDTSKNQVFLKMNSLQTDDTGTYYCTRDAVYGGYGGWFAYWGQGTLVTVSSAKTTPPSVYPLAPGSAAQTNSMVTLGCLVKGYFPEPVTVTWNSGSLSSGVHTFPAVLESDLYTLSSSVTVPSSTWPSETVTCNVAHPASSTKVDKKIVPRDCGCKPCICTVPEVSSVFIFPPKPKDVLTITLTPKVTCVVVAISKDDPEVQFSWFVDDVEVHTAQTQPREEQFNSTFRSVSELPIMHQDWLNGKEFKCRVNSAAFPAPIEKTISKTKGRPKAPQVYTIPPPKKQMAKDKVSLTCMITDFFPEDITVEWQWNGQPAENYKNTQPIMKTDGSYFVYSKLNVQKSNWEAGNTFTCSVLHEGLHNHHTEKSLSHSP

SEQ ID NO: 6 - protein, 216 aa, Mus musculus
DTVLTQSPALAVSLGQRVTISCKASETVSSSMYSYIHWYQQKPGQQPKLLIYRASNLESGVPARFSGSGSGTDFTLTIDPVEADDVATYFCQQSWNPWTFGGGTKLELKRADAAPTVSIFPPSSEQLTSGGASVVCFLNNFYPKDINVKWKIDGSERQNGVLNSWTDQDSKDSTYSMSSTLTLTKDEYERHNSYTCEATHKTSTSPIVKSFNRNEC

SEQ ID NO: 7 - protein, 15 aa, Artificial Sequence
  Synthetic linker
GGGGSGGGGSGGGGS

SEQ ID NO: 8 - protein, 673 aa, Artificial Sequence
  mAb 4H2 fusion to mCD25 variant a
QVQLKESGPGLVQPSQTLSLTCTVSGFSLTSYNVHWVRQPPGKGLEWMGGMRYNEDTSYNSALKSRLSISRDTSKNQVFLKMNSLQTDDTGTYYCTRDAVYGGYGGWFAYWGQGTLVTVSSAKTTPPSVYPLAPGSAAQTNSMVTLGCLVKGYFPEPVTVTWNSGSLSSGVHTFPAVLESDLYTLSSSVTVPSSTWPSETVTCNVAHPASSTKVDKKIVPRDCGCKPCICTVPEVSSVFIFPPKPKDVLTITLTPKVTCVVVAISKDDPEVQFSWFVDDVEVHTAQTQPREEQFNSTFRSVSELPIMHQDWLNGKEFKCRVNSAAFPAPIEKTISKTKGRPKAPQVYTIPPPKKQMAKDKVSLTCMITDFFPEDITVEWQWNGQPAENYKNTQPIMKTDGSYFVYSKLNVQKSNWEAGNTFTCSVLHEGLHNHHTEKSLSHSPGGGGSGGGGSGGGGSELCLYDPPEVPNATFKALSYKNGTILNCECKRGFRRLKELVYMRCLGNSWSSNCQCTSNSHDKSRKQVTAQLEHQKEQQTTTDMQKPTQSMHQENLTGHCREPPPWKHEDSKRIYHFVEGQSVHYECIPGYKALQRGPAISICKMKCGKTGWTQPQLTCVDEREHHRFLASEESQGSRNSSPESETSCPITTTDFPQPTETTAMTETFVLTMEYK

SEQ ID NO: 9 - protein, 656 aa, Artificial Sequence
  mAb 4H2 fusion to mCD25 variant b
QVQLKESGPGLVQPSQTLSLTCTVSGFSLTSYNVHWVRQPPGKGLEWMGGMRYNEDTSYNSALKSRLSISRDTSKNQVFLKMNSLQTDDTGTYYCTRDAVYGGYGGWFAYWGQGTLVTVSSAKTTPPSVYPLAPGSAAQTNSMVTLGCLVKGYFPEPVTVTWNSGSLSSGVHTFPAVLESDLYTLSSSVTVPSSTWPSETVTCNVAHPASSTKVDKKIVPRDCGCKPCICTVPEVSSVFIFPPKPKDVLTITLTPKVTCVVVAISKDDPEVQFSWFVDDVEVHTAQTQPREEQFNSTFRSVSELPIMHQDWLNGKEFKCRVNSAAFPAPIEKTISKTKGRPKAPQVYTIPPPKKQMAKDKVSLTCMITDFFPEDITVEWQWNGQPAENYKNTQPIMKTDGSYFVYSKLNVQKSNWEAGNTFTCSVLHEGLHNHHTEKSLSHSPGGGGSGGGGSGGGGSELCLYDPPEVPNATFKALSYKNGTILNCECKRGFRRLKELVYMRCLGNSWSSNCQCTSNSHDKSRKQVTAQLEHQKEQQTTTDMQKPTQSMHQENLTGHCREPPPWKHEDSKRIYHFVEGQSVHYECIPGYKALQRGPAISICKMKCGKTGWTQPQLTCVDEREHHRFLASEESQGSRNSSPESETSCPITTTDFPQP

SEQ ID NO: 10 - protein, 272 aa, Homo sapiens
MDSYLLMWGLLTFIMVPGCQAELCDDDPPEIPHATFKAMAYKEGTMLNCECKRGFRRIKSGSLYMLCTGNSSHSSWDNQCQCTSSATRNTTKQVTPQPEEQKERKTTEMQSPMQPVDQASLPGHCREPPPWENEATERIYHFVVGQMVYYQCVQGYRALHRGPAESVCKMTHGKTRWTQPQLICTGEMETSQFPGEEKPQASPEGRPESETSCLVTTTDFQIQTEMAATMETSIFTTEYQVAVAGCVFLLISVLLLSGLTWQRRQRKSRRTI

SEQ ID NO: 11 - protein, 219 aa, Homo sapiens
ELCDDDPPEIPHATFKAMAYKEGTMLNCECKRGFRRIKSGSLYMLCTGNSSHSSWDNQCQCTSSATRNTTKQVTPQPEEQKERKTTEMQSPMQPVDQASLPGHCREPPPWENEATERIYHFVVGQMVYYQCVQGYRALHRGPAESVCKMTHGKTRWTQPQLICTGEMETSQFPGEEKPQASPEGRPESETSCLVTTTDFQIQTEMAATMETSIFTTEYQ

SEQ ID NO: 12 - protein, 202 aa, Homo sapiens
ELCDDDPPEIPHATFKAMAYKEGTMLNCECKRGFRRIKSGSLYMLCTGNSSHSSWDNQCQCTSSATRNTTKQVTPQPEEQKERKTTEMQSPMQPVDQASLPGHCREPPPWENEATERIYHFVVGQMVYYQCVQGYRALHRGPAESVCKMTHGKTRWTQPQLICTGEMETSQFPGEEKPQASPEGRPESETSCLVTTTDFQIQ

SEQ ID NO: 13 - protein, 107 aa, Homo sapiens
AYKEGTMLNCECKRGFRRIKSGSLYMLCTGNSSHSSWDNQCQCTSSATRNTTKQVTPQPEEQKERKTTEMQSPMQPVDQASLPGHCREPPPWENEATERIYHFVVGQ

SEQ ID NO: 14 - protein, 165 aa, Homo sapiens
ELCDDDPPEIPHATFKAMAYKEGTMLNCECKRGFRRIKSGSLYMLCTGNSSHSSWDNQCQCTSSATRNTTKQVTPQPEEQKERKTTEMQSPMQPVDQASLPGHCREPPPWENEATERIYHFVVGQMVYYQCVQGYRALHRGPAESVCKMTHGKTRWTQPQLICTG

SEQ ID NO: 15 - protein, 124 aa, Artificial Sequence
  human CD25 ECD residues 1 - 124 with C3A change
ELADDDPPEIPHATFKAMAYKEGTMLNCECKRGFRRIKSGSLYMLCTGNSSHSSWDNQCQCTSSATRNTTKQVTPQPEEQKERKTTEMQSPMQPVDQASLPGHCREPPPWENEATERIYHFVVG

SEQ ID NO: 16 - protein, 42 aa, Homo sapiens
GTMLNCECKRGFRRIKSGSLYMLCTGNSSHSSWDNQCQCTSS

SEQ ID NO: 17 - protein, 5 aa, Homo sapiens
NSGMH

SEQ ID NO: 18 - protein, 17 aa, Homo sapiens
VIWYDGSKRYYADSVKG

SEQ ID NO: 19 - protein, 4 aa, Homo sapiens
NDDY

SEQ ID NO: 20 - protein, 11 aa, Homo sapiens
RASQSVSSYLA

SEQ ID NO: 21 - protein, 7 aa, Homo sapiens
DASNRAT

SEQ ID NO: 22 - protein, 9 aa, Homo sapiens
QQSSNWPRT

SEQ ID NO: 23 - protein, 113 aa, Homo sapiens [sequence omitted]

SEQ ID NO: 24 - protein, 107 aa, Homo sapiens [sequence omitted]

SEQ ID NO: 25 - protein, 439 aa, Homo sapiens
QVQLVESGGGVVQPGRSLRLDCKASGITFSNSGMHWVRQAPGKGLEWVAVIWYDGSKRYYADSVKGRFTISRDNSKNTLFLQMNSLRAEDTAVYYCATNDDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLG

SEQ ID NO: 26 - protein, 440 aa, Homo sapiens
QVQLVESGGGVVQPGRSLRLDCKASGITFSNSGMHWVRQAPGKGLEWVAVIWYDGSKRYYADSVKGRFTISRDNSKNTLFLQMNSLRAEDTAVYYCATNDDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 27 - protein, 214 aa, Homo sapiens
EIVLTQSPATLSLSPGERATLSCRASQSVSSYLAWYQQKPGQAPRLLIYDASNRATGIPARFSGSGSGTDFTLTISSLEPEDFAVYYCQQSSNWPRTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 28 - protein, 673 aa, Artificial Sequence
  nivolumab HC fused to hCD25 variant a
QVQLVESGGGVVQPGRSLRLDCKASGITFSNSGMHWVRQAPGKGLEWVAVIWYDGSKRYYADSVKGRFTISRDNSKNTLFLQMNSLRAEDTAVYYCATNDDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGGGGGSGGGGSGGGGSELCDDDPPEIPHATFKAMAYKEGTMLNCECKRGFRRIKSGSLYMLCTGNSSHSSWDNQCQCTSSATRNTTKQVTPQPEEQKERKTTEMQSPMQPVDQASLPGHCREPPPWENEATERIYHFVVGQMVYYQCVQGYRALHRGPAESVCKMTHGKTRWTQPQLICTGEMETSQFPGEEKPQASPEGRPESETSCLVTTTDFQIQTEMAATMETSIFTTEYQ

SEQ ID NO: 29 - protein, 656 aa, Artificial Sequence
  nivolumab HC fused to hCD25 variant b
QVQLVESGGGVVQPGRSLRLDCKASGITFSNSGMHWVRQAPGKGLEWVAVIWYDGSKRYYADSVKGRFTISRDNSKNTLFLQMNSLRAEDTAVYYCATNDDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGGGGGSGGGGSGGGGSELCDDDPPEIPHATFKAMAYKEGTMLNCECKRGFRRIKSGSLYMLCTGNSSHSSWDNQCQCTSSATRNTTKQVTPQPEEQKERKTTEMQSPMQPVDQASLPGHCREPPPWENEATERIYHFVVGQMVYYQCVQGYRALHRGPAESVCKMTHGKTRWTQPQLICTGEMETSQFPGEEKPQASPEGRPESETSCLVTTTDFQIQ

SEQ ID NO: 30 - protein, 619 aa, Artificial Sequence
  nivolumab HC fused to hCD25 variant d
QVQLVESGGGVVQPGRSLRLDCKASGITFSNSGMHWVRQAPGKGLEWVAVIWYDGSKRYYADSVKGRFTISRDNSKNTLFLQMNSLRAEDTAVYYCATNDDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGGGGGSGGGGSGGGGSELCDDDPPEIPHATFKAMAYKEGTMLNCECKRGFRRIKSGSLYMLCTGNSSHSSWDNQCQCTSSATRNTTKQVTPQPEEQKERKTTEMQSPMQPVDQASLPGHCREPPPWENEATERIYHFVVGQMVYYQCVQGYRALHRGPAESVCKMTHGKTRWTQPQLICTG

SEQ ID NO: 31 - protein, 442 aa, Artificial Sequence
  Nivolumab HC hIgG1.3 lacking C-terminal K
QVQLVESGGGVVQPGRSLRLDCKASGITFSNSGMHWVRQAPGKGLEWVAVIWYDGSKRYYADSVKGRFTISRDNSKNTLFLQMNSLRAEDTAVYYCATNDDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPEAEGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 32 - protein, 443 aa, Artificial Sequence
  nivolumab HC hIgG1.3
QVQLVESGGGVVQPGRSLRLDCKASGITFSNSGMHWVRQAPGKGLEWVAVIWYDGSKRYYADSVKGRFTISRDNSKNTLFLQMNSLRAEDTAVYYCATNDDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPEAEGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 33 - protein, 676 aa, Artificial Sequence
  nivolumab HC IgG1.3 fused to hCD25 variant a
QVQLVESGGGVVQPGRSLRLDCKASGITFSNSGMHWVRQAPGKGLEWVAVIWYDGSKRYYADSVKGRFTISRDNSKNTLFLQMNSLRAEDTAVYYCATNDDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPEAEGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGGGGGSGGGGSGGGGSELCDDDPPEIPHATFKAMAYKEGTMLNCECKRGFRRIKSGSLYMLCTGNSSHSSWDNQCQCTSSATRNTTKQVTPQPEEQKERKTTEMQSPMQPVDQASLPGHCREPPPWENEATERIYHFVVGQMVYYQCVQGYRALHRGPAESVCKMTHGKTRWTQPQLICTGEMETSQFPGEEKPQASPEGRPESETSCLVTTTDFQIQTEMAATMETSIFTTEYQ

SEQ ID NO: 34 - protein, 659 aa, Artificial Sequence
  nivolumab HC IgG1.3 fused to hCD25 variant b
QVQLVESGGGVVQPGRSLRLDCKASGITFSNSGMHWVRQAPGKGLEWVAVIWYDGSKRYYADSVKGRFTISRDNSKNTLFLQMNSLRAEDTAVYYCATNDDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPEAEGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGGGGGSGGGGSGGGGSELCDDDPPEIPHATFKAMAYKEGTMLNCECKRGFRRIKSGSLYMLCTGNSSHSSWDNQCQCTSSATRNTTKQVTPQPEEQKERKTTEMQSPMQPVDQASLPGHCREPPPWENEATERIYHFVVGQMVYYQCVQGYRALHRGPAESVCKMTHGKTRWTQPQLICTGEMETSQFPGEEKPQASPEGRPESETSCLVTTTDFQIQ

SEQ ID NO: 35 - protein, 619 aa, Artificial Sequence; identical to SEQ ID NO: 30
  nivolumab HC IgG1.3 fused to hCD25 variant d

SEQ ID NO: 36 - protein, 5 aa, Mus musculus
NYYMY

SEQ ID NO: 37 - protein, 17 aa, Mus musculus
GINPSNGGTNFNEKFKN

SEQ ID NO: 38 - protein, 11 aa, Mus musculus
RDYRFDMGFDY

SEQ ID NO: 39 - protein, 15 aa, Mus musculus
RASKGVSTSGYSYLH

SEQ ID NO: 40 - protein, 7 aa, Mus musculus
LASYLES

SEQ ID NO: 41 - protein, 9 aa, Mus musculus
QHSRDLPLT

SEQ ID NO: 42 - protein, 120 aa, Artificial Sequence [sequence omitted]

SEQ ID NO: 43 - protein, 111 aa, Artificial Sequence [sequence omitted]

SEQ ID NO: 44 - protein, 446 aa, Artificial Sequence
  humanized mouse variable domain with human IgG4 constant domain
QVQLVQSGVEVKKPGASVKVSCKASGYTFTNYYMYWVRQAPGQGLEWMGGINPSNGGTNFNEKFKNRVTLTTDSSTTTAYMELKSLQFDDTAVYYCARRDYRFDMGFDYWGQGTTVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLG

SEQ ID NO: 45 - protein, 447 aa, Artificial Sequence
  humanized mouse variable domain with human IgG4 constant domain
QVQLVQSGVEVKKPGASVKVSCKASGYTFTNYYMYWVRQAPGQGLEWMGGINPSNGGTNFNEKFKNRVTLTTDSSTTTAYMELKSLQFDDTAVYYCARRDYRFDMGFDYWGQGTTVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 46 - protein, 218 aa, Artificial Sequence
  humanized mouse variable domain with human kappa constant domain
EIVLTQSPATLSLSPGERATLSCRASKGVSTSGYSYLHWYQQKPGQAPRLLIYLASYLESGVPARFSGSGSGTDFTLTISSLEPEDFAVYYCQHSRDLPLTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 47 - protein, 680 aa, Artificial Sequence
  pembrolizumab HC fused to hCD25 variant a
QVQLVQSGVEVKKPGASVKVSCKASGYTFTNYYMYWVRQAPGQGLEWMGGINPSNGGTNFNEKFKNRVTLTTDSSTTTAYMELKSLQFDDTAVYYCARRDYRFDMGFDYWGQGTTVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGGGGGSGGGGSGGGGSELCDDDPPEIPHATFKAMAYKEGTMLNCECKRGFRRIKSGSLYMLCTGNSSHSSWDNQCQCTSSATRNTTKQVTPQPEEQKERKTTEMQSPMQPVDQASLPGHCREPPPWENEATERIYHFVVGQMVYYQCVQGYRALHRGPAESVCKMTHGKTRWTQPQLICTGEMETSQFPGEEKPQASPEGRPESETSCLVTTTDFQIQTEMAATMETSIFTTEYQ

SEQ ID NO: 48 - protein, 663 aa, Artificial Sequence
  pembrolizumab HC fused to hCD25 variant b
QVQLVQSGVEVKKPGASVKVSCKASGYTFTNYYMYWVRQAPGQGLEWMGGINPSNGGTNFNEKFKNRVTLTTDSSTTTAYMELKSLQFDDTAVYYCARRDYRFDMGFDYWGQGTTVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGGGGGSGGGGSGGGGSELCDDDPPEIPHATFKAMAYKEGTMLNCECKRGFRRIKSGSLYMLCTGNSSHSSWDNQCQCTSSATRNTTKQVTPQPEEQKERKTTEMQSPMQPVDQASLPGHCREPPPWENEATERIYHFVVGQMVYYQCVQGYRALHRGPAESVCKMTHGKTRWTQPQLICTGEMETSQFPGEEKPQASPEGRPESETSCLVTTTDFQIQ

SEQ ID NO: 49 - protein, 626 aa, Artificial Sequence
  pembrolizumab HC fused to hCD25 variant d
QVQLVQSGVEVKKPGASVKVSCKASGYTFTNYYMYWVRQAPGQGLEWMGGINPSNGGTNFNEKFKNRVTLTTDSSTTTAYMELKSLQFDDTAVYYCARRDYRFDMGFDYWGQGTTVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGGGGGSGGGGSGGGGSELCDDDPPEIPHATFKAMAYKEGTMLNCECKRGFRRIKSGSLYMLCTGNSSHSSWDNQCQCTSSATRNTTKQVTPQPEEQKERKTTEMQSPMQPVDQASLPGHCREPPPWENEATERIYHFVVGQMVYYQCVQGYRALHRGPAESVCKMTHGKTRWTQPQLICTG

SEQ ID NO: 50 - protein, 449 aa, Artificial Sequence
  pembrolizumab HC hIgG1.3 lacking C terminal K
QVQLVQSGVEVKKPGASVKVSCKASGYTFTNYYMYWVRQAPGQGLEWMGGINPSNGGTNFNEKFKNRVTLTTDSSTTTAYMELKSLQFDDTAVYYCARRDYRFDMGFDYWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPEAEGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 51 - protein, 450 aa, Artificial Sequence
  pembrolizumab HC hIgG1.3
QVQLVQSGVEVKKPGASVKVSCKASGYTFTNYYMYWVRQAPGQGLEWMGGINPSNGGTNFNEKFKNRVTLTTDSSTTTAYMELKSLQFDDTAVYYCARRDYRFDMGFDYWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPEAEGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 52 - protein, 683 aa, Artificial Sequence
  pembrolizumab HC hIgG1.3 hCD25 variant a fusion
QVQLVQSGVEVKKPGASVKVSCKASGYTFTNYYMYWVRQAPGQGLEWMGGINPSNGGTNFNEKFKNRVTLTTDSSTTTAYMELKSLQFDDTAVYYCARRDYRFDMGFDYWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPEAEGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGGGGGSGGGGSGGGGSELCDDDPPEIPHATFKAMAYKEGTMLNCECKRGFRRIKSGSLYMLCTGNSSHSSWDNQCQCTSSATRNTTKQVTPQPEEQKERKTTEMQSPMQPVDQASLPGHCREPPPWENEATERIYHFVVGQMVYYQCVQGYRALHRGPAESVCKMTHGKTRWTQPQLICTGEMETSQFPGEEKPQASPEGRPESETSCLVTTTDFQIQTEMAATMETSIFTTEYQ

SEQ ID NO: 53 - protein, 666 aa, Artificial Sequence
  pembrolizumab HC hIgG1.3 hCD25 variant b fusion
QVQLVQSGVEVKKPGASVKVSCKASGYTFTNYYMYWVRQAPGQGLEWMGGINPSNGGTNFNEKFKNRVTLTTDSSTTTAYMELKSLQFDDTAVYYCARRDYRFDMGFDYWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPEAEGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGGGGGSGGGGSGGGGSELCDDDPPEIPHATFKAMAYKEGTMLNCECKRGFRRIKSGSLYMLCTGNSSHSSWDNQCQCTSSATRNTTKQVTPQPEEQKERKTTEMQSPMQPVDQASLPGHCREPPPWENEATERIYHFVVGQMVYYQCVQGYRALHRGPAESVCKMTHGKTRWTQPQLICTGEMETSQFPGEEKPQASPEGRPESETSCLVTTTDFQIQ

SEQ ID NO: 54 - protein, 629 aa, Artificial Sequence
  pembrolizumab HC hIgG1.3 hCD25 variant d fusion
QVQLVQSGVEVKKPGASVKVSCKASGYTFTNYYMYWVRQAPGQGLEWMGGINPSNGGTNFNEKFKNRVTLTTDSSTTTAYMELKSLQFDDTAVYYCARRDYRFDMGFDYWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPEAEGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGGGGGSGGGGSGGGGSELCDDDPPEIPHATFKAMAYKEGTMLNCECKRGFRRIKSGSLYMLCTGNSSHSSWDNQCQCTSSATRNTTKQVTPQPEEQKERKTTEMQSPMQPVDQASLPGHCREPPPWENEATERIYHFVVGQMVYYQCVQGYRALHRGPAESVCKMTHGKTRWTQPQLICTG